(19) United States
(12) Patent Application Publication
(10) Pub. No.: US 2024/0280574 A1
(43) Pub. Date: Aug. 22, 2024

(54) RAPID RECOGNITION BASED DIAGNOSIS AND TREATMENT

(71) Applicant: Michael L. MARTINO, Pittsburgh, PA (US)

(72) Inventor: Michael L. MARTINO, Pittsburgh, PA (US)

(21) Appl. No.: 18/567,943

(22) PCT Filed: Jun. 7, 2022
(86) PCT No.: PCT/US2022/032557

§ 371 Date: Dec. 7, 2023

Related U.S. Application Data
(60) Provisional application No. 63/197,954, filed on Jun. 7, 2021.

(51) Int. Cl. G01N 33/569

(52) U.S. Cl. CPC G01N 33/56911; G01N 2469/10

(57) ABSTRACT

The current disclosure provides Recognition Based Diagnosis and Treatment (RB-DT) for use in sepsis, toxic Ingestions, and bioterrorism events. The RB-DT includes antibody fragment DT (Fab-DT), Nucleic Acid DT (NA-DT), and covalently bonding antitoxins (AT-DT).

### FIELD OF THE DISCLOSURE

This disclosure relates to a novel, rapid Recognition Based Diagnosis and Treatment (RB-DT) platform to address sepsis, toxic ingestions/exposures and bioterrorism events.

The disclosure describes three methods of employing RB-DT, namely whole or fragments of antibodies (Fab-DT), nucleic acids (NA-DT), and covalently bonding antitoxins (AT-DT). The disclosure further relates to novel methods for accomplishing Fab-DT, including Modified Competitive Elisa (MC-ELISA) and Multiplex Lateral Flow Device (MLFD) for use in surveillance and diagnosis to drive treatment as part of the RB-DT Platform.

### BACKGROUND OF THE DISCLOSURE

Infectious agent and toxin exposures can each be immediately life threatening. Rapid identification of the offending agent and a specific antidote are crucial in determining the source, scope, and remedy of the event. Infectious agent exposure can occur via natural infection (i.e., spontaneous from the internal or external environment of the host subject) or result from intentional, nefarious exposure via aerosolization, water contamination, or other method of biological or chemical terrorism. Likewise, chemical toxins or biologically-derived toxins can be delivered in an act of terrorism, or can be purposefully or accidentally ingested by an individual in an attempted act of self-harm, attempted intoxication, or as an accident (e.g., mistaking a medicine for candy). Current methods for detecting infectious agent and toxin exposures are limited in scope and are characterized by significant time to results. The current disclosure addresses both of these limitations related to infectious agent and toxin exposure.

### SUMMARY OF THE DISCLOSURE

The disclosure provides a Recognition Based Diagnosis and Treatment (RB-DT) platform for use in detecting the presence of antigen exposure and/or toxin infection. The disclosure also provides methods for identifying the source, scope, and remedy of the event as well as providing treatment modalities

In one aspect of the disclosure is a platform for use in treating the presence of toxins or infectious agents in a sample, wherein the sample is a human patient or an environmental sample suspected of having a toxin or infectious agent; using a treatment modality in an assay for detecting the presence of toxins or infectious agents, wherein an amount of the toxin or infectious agent is capable of being quantified; and administering the detected treatment modality when the toxin or infectious agent is detected.

In a second aspect, example embodiments provide an assay system for detecting the presence of toxins in a sample, comprised of an assay plate, a population of toxin specific primary antibodies, and a secondary antibody, wherein the population of toxin specific primary antibodies are monoclonal; a single, toxin specific primary antibody from the population of antibodies is affixed to a single well of the assay plate and the secondary antibody is specific to heavy chain constant regions of each of the toxin specific primary antibodies and the binding is capable of being quantified.

In a third aspect, example embodiments provide an article for detecting the presence of toxins in a sample, comprising a population of toxin specific primary antibodies, affixed to an assay plate, wherein the population of toxin specific primary antibodies are monoclonal; and a single, toxin specific primary antibody from the population of antibodies is affixed to a single well of the assay plate via PEGylation.

In a fourth aspect, example embodiments provide an a method for detecting the presence of a toxin in a sample comprising preparing a sample; passing the sample over an assay plate comprised of toxin specific primary antibodies, affixed to the assay plate; agitating the assay plate; removing the sample; adding a secondary antibody with a quantifiable label and that specifically binds to the heavy chain constant region of the primary antibody; agitating the assay plate; removing the secondary antibody; and quantifying the binding of the secondary antibody to the heavy chain constant region wherein secondary antibody binding to the heavy chain constant region is inversely proportional to the quantity of toxin in the sample.

In a fifth aspect, example embodiments provide a lateral flow device for detecting the presence of toxins in a sample, wherein the lateral flow device is comprised of an absorbent pad, nitrocellulose membrane, and antibody coated latex microbeads.

In a sixth aspect, example embodiments provide an assay system for detecting the presence of toxins in a sample, comprised of an assay plate, a population of toxin specific nucleic acid primers, and associated probes, wherein the population of toxin specific nucleic acid primers are directed against sequences of an infecting agent or toxin; polymerase chain reaction is use to amplify the sequence in a sample of interest and the sequence of an infecting agent or toxin is capable of being quantified.

In a seventh aspect, example embodiments provide an assay system for detecting the presence of toxins in a sample, comprised of an assay plate and a population of antitoxins, wherein the population of antitoxins covalently bind a toxin in a sample; and the binding between antitoxins and toxins is capable of being quantified.

Other aspects of the disclosure provide a novel lateral flow device and a kit comprising a novel type of ELISA (MC-ELISA) for detecting the presence of a toxin or infectious agent.

Specific embodiments of the disclosure will become evident from the following more detailed description and the claims.

### BRIEF DESCRIPTION OF THE DRAWINGS

FIG. 1 is a schematic of Hierarchy of Recognition-Based Diagnosis & Treatment of the RB-DT paradigm, its three derivatives (Fab-DT, NA-DT, and AT-DT), novel machinations to accomplish the proposed Fab-DT which are Modified Competitive ELISA (MC-ELISA) and Multiplex Lateral Flow Device (MLFD). The schematic also provides an overview of RB-DT's potential for surveillance.

FIG. 2 is a schematic of antibody fragment production and assays including Enzyme-Linked Immunosorbent Assay (ELISA) and Lateral Flow Devices (LFD) as well as the use of Fab-Fragments and Other Antibody Fragments for use in treatment.

FIG. 3 is a schematic of traditional types of Enzyme-Linked Immunosorbent Assays (ELISA).

FIG. 4 is a block diagram overview of the proposed novel Modified Competitive Enzyme-Linked Immunosorbent Assay (MC-ELISA) method.

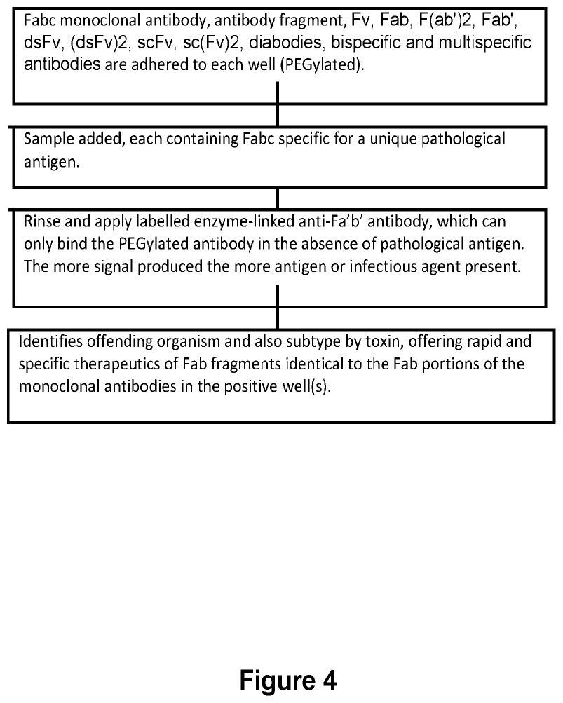

FIG. 5 is a schematic of the of the modified competitive bioassay. An antibody or antibody fragment specific for an antigen is affixed, via PEGylation or other methods to one or more wells of an assay plate or chamber of a lateral flow device (FIG. 5A). A prepared sample is then added to wells containing the antibody or antibody fragments. If the antigen or infectious agent of interest is present in the sample then it binds to the affixed antibody (fragments). The antigen fails to bind in wells coated with antibody (fragments) possessing other antigenic specificities (FIG. 5B). A secondary enzyme-linked antibody possessing affinity for the primary antibody's Light Chain (LC) constant region is added to the well. The secondary antibody only binds primary antibodies which are themselves unbound to antigen. The result is a quantifiable change that can either be visualized and/or measured photometrically (FIG. 5C).

FIG. 6 is a schematic of Illustration of Light Chain Binding Sites for MC-ELISA Secondary Antibody

FIG. 7 is a schematic of a multiplex lateral flow device (MLFD).

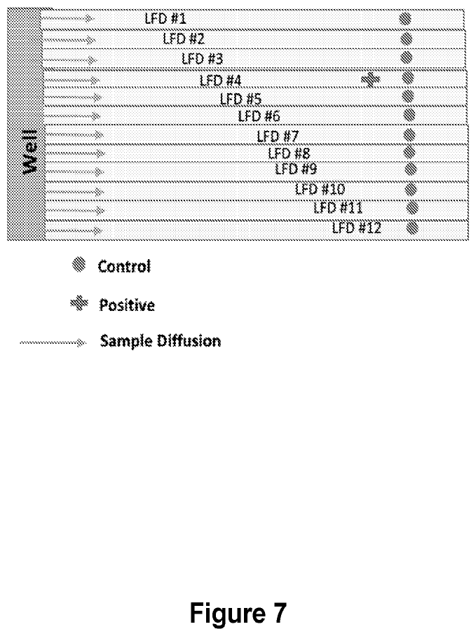

FIG. 8 is a schematic of a multiplex lateral flow device (MLFD) for continuous environmental sampling. A humidifier is connected to the continuous environmental sample input. A rotating fan draws an environmental sample through the well, creating a turbulent flow within the multi-lateral flow device. As a result, environmental samples are pulled through the well where they enter the lateral flow chambers (LFD #1-#12) coated with antibodies to a toxin or infectious agent, with the output read by an optical detector using a laser.

### DETAILED DESCRIPTION OF THE PREFERRED EMBODIMENT

The disclosure provides Recognition Based Diagnosis and Treatment (RB-DT) for use in addressing sepsis, toxic ingestions/exposures and bioterrorism events. The disclosure describes systems, methods, lateral flow devices, and kits for rapid identification of infectious and bioterrorism agents. The Rb-DT, as disclosed herein also provides methods for antibody-driven diagnosis in combination with antibody and/or antibody fragment-driven treatment.

Reference will now be made in detail to exemplary embodiments of the claimed invention. While the claimed invention will be described in conjunction with the exemplary embodiments, it will be understood that it is not intended to limit the claimed invention to those embodiments. To the contrary, it is intended to cover alternatives, modifications, and equivalents, as may be included within the spirit and scope of the claimed invention, as defined by the appended claims.

Those of ordinary skill in the art may make modifications and variations to the embodiments described herein without departing from the spirit or scope of the claimed invention. In addition, although certain methods and materials are described herein, other methods and materials that are similar or equivalent to those described herein can also be used to practice the claimed invention.

In addition, any of the compositions or methods provided, disclosed, or described herein can be combined with one or more of any of the other compositions and methods provided, disclosed, or described herein.

### 1. Definitions

Unless defined otherwise, all technical and scientific terms used herein have the meaning commonly understood by a person skilled in the art to which the claimed invention belongs. The terminology used herein is for describing particular embodiments only and is not intended to be limiting of the claimed invention. All technical and scientific terms used herein have the same meaning.

The following references provide those of skill in the art with a general understanding of many of the terms used herein (unless defined otherwise herein): Singleton et al., Dictionary of Microbiology and Molecular Biology, 3rd ed. (Wiley, 2006): Walker, The Cambridge Dictionary of Science and Technology (Cambridge University Press, 1990); Rieger et al., Glossary of Genetics: Classical and Molecular, 5th ed. (Springer Veriag, 1991); and Hale et al., Harper Collins Dictionary of Biology (HarperCollins Publishers, 1991). Standard techniques, known in the art, can be found in reference manuals such as, for example, Green et al., Molecular Cloning: A Laboratory Manual, 4th ed. (Cold Spring Harbor Laboratory Press, 2012), and Ausubel, Current Protocols in Molecular Biology (John Wiley & Sons Inc., 2004).

The following terms may have meanings ascribed to them below, unless specified otherwise. However, it should be understood that other meanings known or understood by those having ordinary skill in the art are also possible, and within the scope of the claimed invention. All publications, patent applications, patents, and other references mentioned or discussed herein are expressly incorporated by reference in their entireties. In the case of conflict, the present specification, including definitions, will control. In addition, the materials, methods, and examples are illustrative only and not intended to be limiting.

As used herein, the singular forms “a,” “and,” and “the” include plural references, unless the context clearly dictates otherwise.

As used herein, the term “or” means, and is used interchangeably with, the term “and/or,” unless context clearly indicates otherwise.

As used herein, the term “including” means, and is used interchangeably with, the phrase “including but not limited to.”

As used herein, the term “such as” means, and is used interchangeably with, the phrase “such as, for example” or “such as but not limited.”

Unless specifically stated or obvious from context, as used herein, the term “about” is understood as within a range of normal tolerance in the art, for example, within two standard deviations of the mean. About can be understood as within 10%, 9%, 8%, 7%, 6%, 5%, 4%, 3%, 2%. 1%, 0.5%, 0.1%, 0.05%, or 0.01% of the stated value. Unless otherwise clear from context, all numerical values provided herein can be modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the term “assay system” refers to an assay and all needed components to conduct the assay. Assay systems can include assay plates containing between 2-wells and 256 wells, such as 6-well, 12-well, 24-well, 36-well, 48-well, 60-well, 72-well, 84-well, 96-well, or 256-well. Assays systems can also include additional components that may be required for performing an assay including, but not limited to, buffers, reagents, positive and negative controls, membranes, vessels, tubes, sealing tapes, and instructions. The assay system may be comprised of a single or a population of toxin specific and/or infectious disease specific primary antibodies, and a secondary antibody. In a preferred embodiment the population of toxin specific primary antibodies are monoclonal.

In another embodiment the assay system may contain an antibody or fragments thereof. including Fab, (Fab′2), or fragments thereof (Fv, Fab, F(ab′)2, Fab′, dsFv, (dsFv)2, scFv, sc(Fv)2, diabodies). The antibodies can also be humanized and detect proteins post-translationally modified following interaction with the antigen or infectious agent. The secondary antibody is any secondary antibody that binds to the one or more primary antibodies. In one embodiment the secondary antibody contains a colorimetric label that that can be visualized to indicate the presence or absence of a toxin or infectious agent. In a further embodiment the secondary label can be fluorescent, radioactive, or enzymatic-based. In one embodiment the secondary label can be visualized. In another embodiment the label can be quantified. In a preferred embodiment of the disclosure the antibodies are attached to the plate using PEGylation.

The term “bacteria” as used herein are single-celled prokaryotic organisms with a definite cell wall. The term “fungi” as used herein are unicellular or multicellular, eukaryotic organisms having no chlorophyll. Several fungal species are known to cause diseases in humans. The term “toxins” as used herein are secondary metabolites produced by bacteria, fungi, algae, plants, fishes, crustaceans, and mollusks and are known to act in very low concentrations and can affect the functioning of cells. Each of these agents can independently be a toxin or infectious agent or can be modified for the same purpose.

As used herein, the term “toxin” or “biotoxin” refers to harmful substances produced within a living cell or organism. Toxins can be of plant or animal origin and encompass chemical, biological, physical, radiation and behavioral toxicity. Biotoxins include those toxins with a biological (i.e., plant or animal) origin and can originate from spiders, snakes, scorpion, jellyfish, wasps, bees, ants, termites, spiders, and wasps, among others.

Examples of biotoxins include, but are not limited to cyanotoxins, dinotoxins, necrotoxins, neurotoxins, myotoxins, and cytotoxins. Examples of toxins include, but are not limited to botulinum toxin A (from bacteria Clostridium botulinum), tetanus toxin A (from Clostridium tetani), diphtheria toxin (from Corynebacterium diphtheriae), muscarine (from Amanita muscaria), bufotoxin (from genus Bufo), ricin, Staphyloccous aureus, and trichothecene (fungus). Other bacterial toxins include Endotoxin A (Pseudomonas), Exoenzyme S, Shiga Toxins STX-1 and STX-2 (Enterohemorrhagic E. coli), RTXA1 toxin (Vibrio vulnificans), Staph aureus, Toxic Shock Syndrome-1, Scalded Skin Syndrome, and Exotoxins A and B. As used herein, the term “toxin” may also refer to manufactured, synthetic, and environmental substances. Examples include dioxin, sarin, polychlorinated biophenyls (PCBs), metals and heavy metals such as arsenic, lead, mercury, cadmium, chromium, and pesticides such as dicholordiphenyltrichloroethane (DDT) and sulfuryl fluoride.

As used herein, the term “Infectious agents” refers to organisms that are capable of producing an infection or infectious disease. Infectious agents include bacteria, archaea, amoebae, fungi, viruses, and parasites. Infectious agents are often broadly categorized into six main classes including prions, viruses, bacteria, fungi, protozoa, and helminths.

As used herein, the term “antibody” refers to conventional antibodies, single domain antibodies, including heavy chain of single domain antibodies, and chimeric, humanized, bispecific or multi-specific antibodies, and fragments of each. Antibodies may comprise two heavy chains linked to each other by disulfide bonds with each heavy chain linked to a light chain by a disulfide bond. The light chains can be lambda (l) or kappa (k) whereas the heavy chain antibodies, as noted herein can be any of the five main heavy chain classes (IgM, IgD, IgG, IgA and IgE), which determine the functional activity of the antibody. The light chain includes two domains or regions, a variable domain (VL) and a constant domain (CL). The heavy chain is comprised of four domains, a variable domain (VH) and three constant domains (CH1, CH2 and CH3 (i.e., the CH region). The variable light region (VL) and variable heavy region (VH) of an antibody determine binding recognition and specificity to an antigen. The constant region domains of the light (CL) and heavy (CH) chains confer biological properties including binding to Fc receptors (FcR). Complementarity Determining Regions or CDRs refer to amino acid sequences that together define the binding affinity and specificity of the natural Fv region of an antibody binding site.

As used herein, the term “antibody” also includes single domain antibodies which are antibodies with complementary determining regions that are part of a single domain polypeptide. Examples of single domain antibodies include heavy chain antibodies, antibodies naturally devoid of light chains, single domain antibodies derived from conventional four-chain antibodies, and engineered single domain antibodies. Variable heavy chain of single domain antibodies devoid of light chains are known as VHH or nanobodies. Any of these antibodies can be used in the assay systems, lateral flow devices, and related detection systems, as described herein.

As used herein, the term “monoclonal antibody” as used herein refers to an antibody molecule directed against a specific antigen. Monoclonal antibodies are directed to a single antigen and are a preferred embodiment of the current disclosure. “Polyclonal antibodies” can also be used in the current disclosure and are a collection of immunoglobulins against a specific antigen

As used herein, the term “humanized antibody” refers to an antibody which is wholly or partially of non-human origin and which has been modified to replace certain amino acids, in order to avoid or minimize an immune response in humans.

As used herein, the term “fragments” of refers to antibodies that comprise a portion of an intact antibody, in particular the antigen binding region or variable region of the intact antibody that could be used in the assay system, lateral flow devices, and related detection systems described herein. Examples of antibody fragments include Fv, Fab, F(ab′)2, Fab′, dsFv, (dsFv)2, scFv, sc(Fv)2, diabodies, bispecific and multi-specific antibodies originating or formed from antibodies and can also include single domain antibodies, such as a heavy chain antibody or VHH.

As used herein, the term “F(ab′)2” refers to an antibody fragment having a molecular weight of about 100,000 and antigen binding activity, which is slightly larger than the Fab bound via a disulfide bond of the hinge region.

As used herein, the term “Fab′” refers to an antibody fragment having a molecular weight of about 50,000 and antigen binding activity, which is obtained by cutting a disulfide bond of the hinge region of the F(ab′)2. Fab fragments are devoid of the Fc fragment. An entire native antibody is referred to as a “Fabc”.

As used herein, the term “Fc fragment” refers to the region of an antibody that is constant in a given organism, unique to various species, and antigenic across species. In one embodiment, Fc fragments are advantageous for use in the assay systems described herein, as well as treatment modalities because of the intrinsic characteristics of Fab fragments including: (1) administration without an Fc-driven inflammatory cascade; (2) large scale production in vivo or in vitro from species other than the end recipient, although the current disclosure also includes production in human hosts following infection; (3) their use is with reduced concern for inflammatory reaction driven by recognition of foreign Fc fragment from another organism/species, especially on repeated dosing; (3) and cultivation for specific targeting of a wide variety of antigens, provided sufficient antigen size

As used herein, the term “single chain Fv” (“scFv”) refers to a polypeptide is a covalently linked VH:VL heterodimer which is usually expressed from a gene fusion including VH and VL encoding genes linked by a peptide-encoding linker. scFvs, or can be generated by coupling monovalent scFvs by a peptide linker.

Post translational modifications refers to the covalent and generally enzymatic modification of proteins following protein biosynthesis. Post-translational modifications can occur on the amino acid side chains or at the protein's C- or N-termini. Post translational modifications include over 400 different modifications. Common post-translational modifications include, but are not limited to phosphorylation, glycosylation, ubiquitination, nitrosylation, methylation, hydroxylation, AMPylation, acetylation, lipidation, Ubiquitination, biotinylation, glycylation, isoprenylation, sulfation, and proteolysis. The post translational modifications can change the function and properties of a protein. The assay system, lateral flow device, and detection system of the instant disclosure offers the ability to detect proteins using antibodies that detect proteins of interest with or without post-translational modifications

As used herein, the term “pathogen” or “infectious agent” or “germ” refers to viruses, bacteria, fungi, and protozoa. Such pathogens, when used in biological warfare or as used as bioterrorism agents can give rise to disease in man and animals, when intentionally (or accidentally) released. Such agents can rapidly cause large-scale mortality, morbidity, and/or incapacitation of a large number of people with adverse effects on human health. The effects of pathogens, as used herein, includes pathogens that may result in near instantaneous onset of symptoms to those pathogens in which symptoms onset may require hours or weeks. Pathogens can be released in small quantities and are capable of self-replication either independently or dependent upon a cellular host. Pathogens used as bioterrorism agents, which can be detected by the invention of current disclosure are divided into categories A-C. Bioterrorism agents of category A are high priority agents that can be easily disseminated or transmitted from person to person; result in high mortality rates and have the potential for major public health impact; might cause public panic and social disruption; and require special action for public health preparedness. Examples include Anthrax (Bacillus anthracis), Botulism (Clostridium botulinum toxin). Plague (Yersinia pestis), Smallpox (variola major), Tularemia (Francisella tularensis), Viral hemorrhagic fevers, including Filoviruses (Ebola, Marburg) and Arenaviruses (Lassa, Machupo). Category B bioterrorism agents are easy to disseminate and have a moderate morbidity and mortality rate, as defined by the CDC. Category B bioterrorism agents include Brucellosis (Brucella species), Epsilon toxin of Clostridium perfringens, Food safety threats (Salmonella species, Escherichia coli O157:H7, Shigella), Glanders (Burkholderia mallei), Melioidosis (Burkholderia pseudomallei), Psittacosis (Chlamydia psittaci), Q fever (Coxiella burnetii), Ricin toxin from Ricinus communis (castor beans), Staphylococcal enterotoxin B, Typhus fever (Rickettsia prowazekii), Viral encephalitis (alphaviruses, such as eastern equine encephalitis, Venezuelan equine encephalitis, and western equine encephalitis]), and Water safety threats (Vibrio cholerae, Cryptosporidium parvum). Category C bioterrorism agents are those pathogens that could be engineered for mass dissemination because they have high availability, ease of production and potential for high morbidity and mortality.

In addition, infectious organisms cause disease by releasing toxins and bacterial components which are toxic to the host, binding to specific host receptors and thereby augmenting or inhibiting normal physiologic processes. Bacteria may produce exoenzymes which catalyze reactions to disrupt immune responses and/or homeostasis. Without toxic substances, colonization, or in some cases even symbiosis, would occur but without disease. Consistent with this, infection, as opposed to colonization, requires the presence of bacterial toxins and infections may be considered poisonings with the specific bacterial toxin(s), which is exploited in the current disclosure to rapidly identify the infecting agent or toxin and the treatment modality.

The term “virus or viruses” as used herein are capable of replication inside a living cell and are pathogenic to humans and animals. Viruses also include emerging and novel viruses, such as the SARS-CoV-2 and other known and emerging coronaviruses.

As used herein, viruses are comprised of proteins and nucleic acids with the ability to multiply and spread very quickly. Viruses include, but are not limited to Variola virus, Ebola virus, Marburg virus,

### 2. Sepsis

As used herein, the term “sepsis” refers to a biological response to an infectious agent in a human characterized by one or more of tachycardia, fever, elevated white count and tachypnea as signs of an immune response”. Sepsis can progress to Severe Sepsis with dysfunction of one or more organs and/or elevated lactate (indicating poor perfusion and/or mitochondrial dysfunction with relative uncoupling of oxidative respiration), and then finally to Septic Shock, with very elevated lactate levels and/or hypotension even after standard iv fluid administration.

Each stage from Sepsis to Severe Sepsis to Septic Shock comes with a greater physiologic response, metabolic expense and probability of mortality to the infected person. Sepsis has a mortality of 10-20%, Severe Sepsis mortality ranges from 20-40% and altogether have Septic Shock has a mortality of 40-80% (Singer; Martin). Sepsis is a leading cause of death worldwide. The U.S. is no exception, where over 1 million patients with sepsis are admitted annually (Angus), and account for 50% of all hospital deaths (Liu). In 2013, sepsis accounted for more than $24 billion in hospital expenses in the US alone, representing 13% of total U.S. hospital costs (Torio).

The global burden of sepsis is difficult to ascertain, although a recent scientific publication estimated that in 2017 there were 48.9 million cases of sepsis and 11 million sepsis-related deaths worldwide (Rudd). Sepsis accounts for nearly 20% of all global deaths (Singer). Immunosuppressed people, and those with significant comorbidities, malnutrition, indwelling medical devices, or recent surgical procedures are at particular risk of developing, and dying from, sepsis. At-risk populations include older persons, pregnant or recently pregnant women, neonates, hospitalized patients, intensive care unit patients, HIV/AIDS patients, individuals with liver cirrhosis, cancer patients, individuals with kidney disease, people with autoimmune diseases, people with no spleen, organ transplant recipients, impoverished people with poor nutrition and poor medical access.

Bacteria mediate sepsis by means of bacterial toxins, which bind to specific host targets, eliciting tissue damage, malfunction, and inflammatory response. Some toxins are shared by many bacteria, such as LPS (lipopolysaccharide, a cell membrane component of Gram-negative bacteria). LPS is shared by infecting, colonizing and symbiotic bacteria. It is the release of LPS due to inflammation and bacterial destruction which liberates it as a toxin. Thus, LPS has remained an elusive pharmacologic target.

In contrast, the most virulent bacteria species, responsible for the greatest numbers of sepsis and resultant deaths, each have specific unique bacterial toxins which promote infection and inflammation. Each of these unique bacterial toxins could be used to identify the bacterial source, and can serve as pharmacologic targets for amelioration of sepsis (see Table 1).

TABLE 1
Unique Bacterial Toxins: Disease-
Causing Agents and RB-DT Targets

| Organism | Toxins |
|---|---|
| Pseudomonas | Exotoxin A* |
| | Exoenzyme S |
| Enterohemorrhagic E. coli | Shiga Toxins STX 1 and STX 2 |
| Vibrio vulnificans | RTXA1 toxin |
| Staph aureus | |
| Toxic Shock Syndrome | TSST-1 |
| Scalded Skin Syndrome | Endotoxins A and B* |
| Clostridium difficile | Enterotoxin A* |
| | Enterotoxin B* |
| Clostridium botulinum | Toxins A*, B*, C, D, E, F, G, H (one or |
| | more present in various subspecies) |

### 3. Toxic Ingestions

Poisoning is a significant problem in the United States and is the leading cause of unintentional injury death, surpassing motor vehicle crashes. In 2010, fatalities from unintentional poisoning totaled 33,041 (CDC). Approximately 2.3 million unintentional poisonings or poison exposures (predominately nonfatal) were reported to poison control centers in 2011 (Mowry).

Across all ages, 76.9% of poison exposures reported to U.S. poison centers in 2020 were unintentional, 18.3% were intentional, and 2.7% were adverse reactions. In children younger than 6 years, 99.2% of exposures are unintentional, compared to only 31.4% of teen exposures and 63.2% of adult exposures (See Table 2).

TABLE 2
Toxicology and Reasons for Human Exposure
Cases by Age Group (2020)

| | <6 | 6-12 | | | All |
|---|---|---|---|---|---|
| | Years | Years | Teens | Adults | Ages |
| Unintentional | | | | | |
| General | 92.1% | 41.8% | 9.1% | 14.7% | 48.3% |
| Environmental | 0.7% | 3.3% | 2.1% | 4.9% | 2.9% |
| Occupational | 0.0% | 0.0% | 0.9% | 2.6% | 1.2% |
| Therapeutic error | 5.0% | 22.3% | 9.4% | 20.3% | 13.0% |
| Misuse | 0.6% | 11.8% | 7.3% | 16.0% | 8.6% |
| Bite/sting | 0.5% | 3.1% | 1.7% | 2.9% | 1.8% |
| Food poisoning | 0.3% | 1.1% | 0.7% | 1.5% | 0.9% |
| Unknown | 0.0% | 0.2% | 0.2% | 0.4% | 0.2% |
| Subtotal | 99.2% | 83.6% | 31.4% | 63.2% | 76.9% |
| Unintentional | | | | | |
| Intentional | | | | | |
| Intentional - | 0.0% | 5.5% | 50.7% | 18.5% | 12.5% |
| Suspected | | | | | |
| suicide | | | | | |
| Intentional - | 0.0% | 5.8% | 5.3% | 4.1% | 2.6% |
| Misuse | | | | | |
| Intentional - | 0.0% | 0.4% | 6.1% | 4.1% | 2.3% |
| Abuse | | | | | |
| Intentional - | 0.0% | 0.9% | 2.2% | 1.4% | 0.9% |
| Unknown | | | | | |
| Subtotal | 0.0% | 12.6% | 64.3% | 28.0% | 18.3% |
| Intentional | | | | | |
| Adverse | 0.3% | 1.8% | 2.2% | 5.2% | 2.7% |
| Reaction | | | | | |
| Other/Unknown | 0.5% | 2.0% | 2.2% | 3.5% | 2.1% |

Data from National Capital Poison Center

### 4. Bioterrorism

As used herein, “Bioterrorism” is the use of any biological or chemical agent for an attack on a multitude of people. Bioterrorism events have the potential to affect thousands of people simultaneously. Bioterrorism weapons can be utilized in a military theater, at a public gathering (e.g., a sports stadium), in a public transport system, or in a municipal water supply, among others. These attacks can be difficult to detect, with the potential to produce an overwhelming number of casualties with little warning, quickly surpassing local medical capacity. Medical care is often only supportive, as few specific antidotes are available.

As used herein, the term “biologic agents of concern” includes 29 biological agents identified by the CDC as of concern because they could be utilized in bioterrorism The CDC details 29 biological agents of concern which could be utilized in bioterrorism. These are divided into three categories based on likelihood and ease of weaponization (see Table 3).

TABLE 3
Biologic Agents of Bioterrorism

| | Disease | Agent |
|---|---|---|
| Category A | Anthrax | Bacillus anthracis |
| | Botulism | Clostridium botulinum toxin |
| | Plague | Yersinia pestis |
| | Smallpox | Variola major |
| | Tularemia | Francisella tularensis |
| | Viral hemorrhagic | Ebola virus |
| | fevers | |
| | | Marburg virus |
| | | Lassa virus |
| | | Machupo virus |
| Category B | Food poisoning | Epsilon toxin of Clostridium perfringes |
| | | Salmonella sp. |
| | | E. coli O157:H7 |
| | | Shigella |
| | Glanders | Burkholderia mallei |
| | Meliodosis | Burholderia pseudomallei |
| | Psittacosis | Chlamydia psittaci |
| | Q fever | Coxiella burnetti |
| | Ricin poisoning | Ricin Toxin of Ricinus communis |
| Category C | Encephalitis | Nipah virus |
| | Hantavirus | Hantavirus |
| | Pulmonary | |
| | Syndrome (HPS) | |

Category A comprises the ten highest priority agents as defined by the following criteria: can be easily disseminated or transmitted from person to person; result in high mortality rates and have potential for major public health impact; might cause public panic and social disruption; and requires special action for public health preparedness.

Category B comprises the next highest priority items as detailed in Table 3 and are characterized in that they are moderately easy to disseminate, result in moderate morbidity rates and low mortality rates; and require specific enhancements of CDC's diagnostic capacity and enhanced disease surveillance. For example [Soligenix website], Ricin toxin is easily obtained from castor beans directly, or as byproduct from castor oil production. Ricin toxin is 6000 times by weight more potent than Cyanide, and can rapidly enter cells within 4 hours. Once cells have been entered, Ricin toxin inhibits ribosome protein synthesis and that cell will die. By 8 hours victims may experience gastrointestinal bleeding and pulmonary edema, followed by multiorgan failure and death within 48 hours. Ricin toxin can be aerosolized, ingested or injected. A fatal dose can fit on a pinhead. “RiVax” is a preventative vaccine containing inactivated Ricin toxin, and given in advance of an exposure, it induces antibody protection against Ricin poisoning. However, induction of antibodies takes several days or more, too late to save a patient with an acute fatal exposure. No antidote currently exists, but the RiVax vaccine proves the Ricin toxin is immunogenic and antibodies (or their fragments) can neutralize the toxin and prevent death, as occurs when RiVax is given in advance.

Category C agents include emerging infectious diseases such as Nipah virus and Hantavirus which could be engineered for mass dissemination in the future because of availability; ease of production and dissemination; and potential for high morbidity and mortality rates and major health impact.

The CDC also notes 61 agents in 10 classes of chemical toxins weaponization, some of which are derived from biological sources (see Table 4). These classes include:

TABLE 4
Chemical Toxin Weaponization

| | |
|---|---|
| Blister agents (Vesicants) | Lewsite (L, L-1, L-2, L-3) |
| | Nitrogen Mustard (HN-1, HN-2, HN-3) |
| | Phosgene oxime (CX) |
| | Sulfur Mustard (H) (mustard gas) |
| Choking/Lung Agents | Ammonia |
| | Osmium tertroxide |
| | Bromine (CA) |
| | Phosgene (CG) |
| | Chlorine (CL) |
| | Phosphine |
| | Hydrogen chloride |
| | Phosphorus (elemental, white or yellow) |
| | Methyl bromide |
| | Sulfuryl fluoride |
| Nerve Agents | Sarin (GB) |
| | Soman (GD) |
| | Tabun (GA) |
| | VX |
| Biotoxins (e.g., | Abrin |
| Ricin, Strychnine) | Ricin |
| | Brevetoxin |
| | Saxitoxin |
| | Colchicine |
| | Strychnine |
| | Digitalis |
| | Tetrodotoxin |
| | Nicotine |
| | Trichothecene |
| Blood Agents (e.g., | Arsine |
| Cyanide) | Carbon Monoxide |
| | Cyanide [Cyanogen chloride (CK), |
| | Hydrogen cyanide (AC), Potassium |
| | cyanide (KCN), Sodium Cyanide (NaCN)] |
| | Sodium monofluoroacetate (compound |
| | 1080) |
| Caustics (Acids) | Hydrofluoric acid |
| Incapacitating Agents | BZ, Fentanyls, other Opioids |
| Metals | Arsenic, Barium, Mercury, Thallium |
| Riot Control Agents/Tear Gas | Bromobenzylcyanide (CA), |
| | Chloroacetophenone (CN), |
| | Chlorobenzylidenemalononitrile (CS), |
| | Chloropicrin (PS), Dibenzoxazepine (CR) |
| Toxic Alcohols | Ethylene glycol |

### 5. Recognition Based Diagnosis and Treatment (RB-DT)

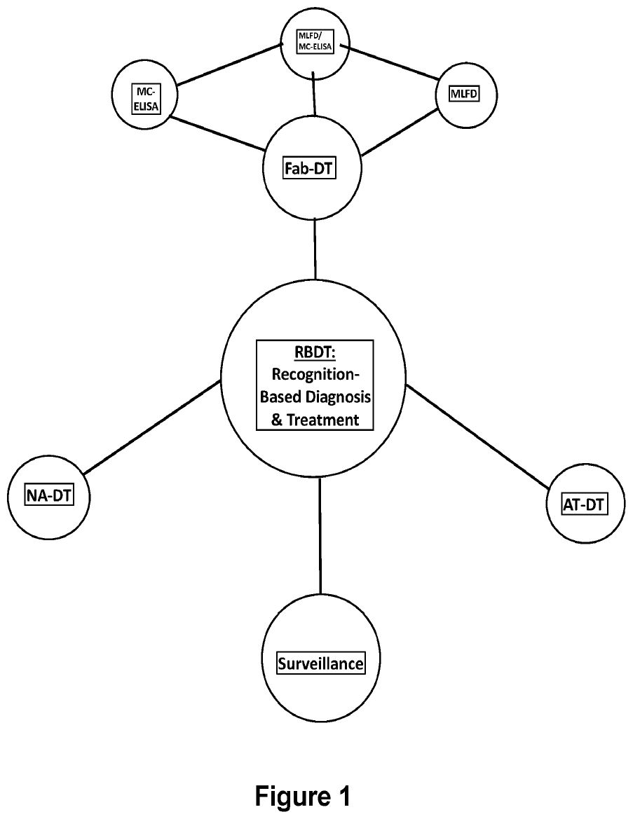

As used herein the term “Recognition Based Diagnosis and Treatment (RB-DT)” refers to a novel, rapid platform to address sepsis from infectious agents, toxicologic exposures, and bioterrorism events. The basis of RB-DT is to derive a rapid and readily available means of positively identifying the offending agent from a large number of similar possibilities, and then immediately utilizing that same instrument of in vitro identification as specific rescue treatment in vivo. Three modalities lend themselves to RB-DT (see FIG. 1).

As used herein the term “AntiToxin-driven Diagnosis and Treatment (AT-DT)” refers to a first consideration for accomplishing RB-DT involving the use of covalently bonding antitoxins. Via specific chemical recognition, an array of covalently bonding antitoxins—either enzymatic or nonenzymatic—can identify one agent from a pool of other potential agents.

The term “Nucleic Acid-driven Diagnosis and Treatment (NA-DT)” as used herein is a second consideration for achieving RB-DT and refers to the use of nucleic acids via Polymerase Chain Reaction (PCR) amplification using Nucleic Acid-driven Diagnosis and Treatment (NA-DT). PCR is a powerful tool for identification of specific sequences of nucleic acids produced by biological entities, e.g., bacteria and viruses. Amplification of predetermined sequences can identify an array of unknowns from a large and diverse biological sample. The same DNA primer used to “fish” for a complementary nucleic acid sequence produced by a biologic entity (DNA or RNA from bacteria, viruses and other microbes) can also be turned about in vivo as a therapeutic. Binding bacterial RNA and preventing protein synthesis is a very effective way to slow infection and reduce the production of specific bacterial toxins.

As used herein, the third consideration for effecting RB-DT is to employ antibodies or their derivative fragments for diagnosis and treatment (Fab-DT). Antibodies, in general have an extremely wide-ranging recognition capacity, while each individual monoclonal or oligoclonal antibody product is very specific to given epitope or small set of epitopes. Antibodies or their various fragments, have the most immediate potential to identify post-translational toxins and infectious agents. Additionally, antibody preparations can be kept stable for an extended period as lyophilized powder for easy storage, reconstitution, and administration to patients. Only where very small molecules serve as toxins with few if any antigenic epitopes, would NA-DT or AT-DT be a preferred strategy over Fab-DT to accomplish the RB-DT platform.

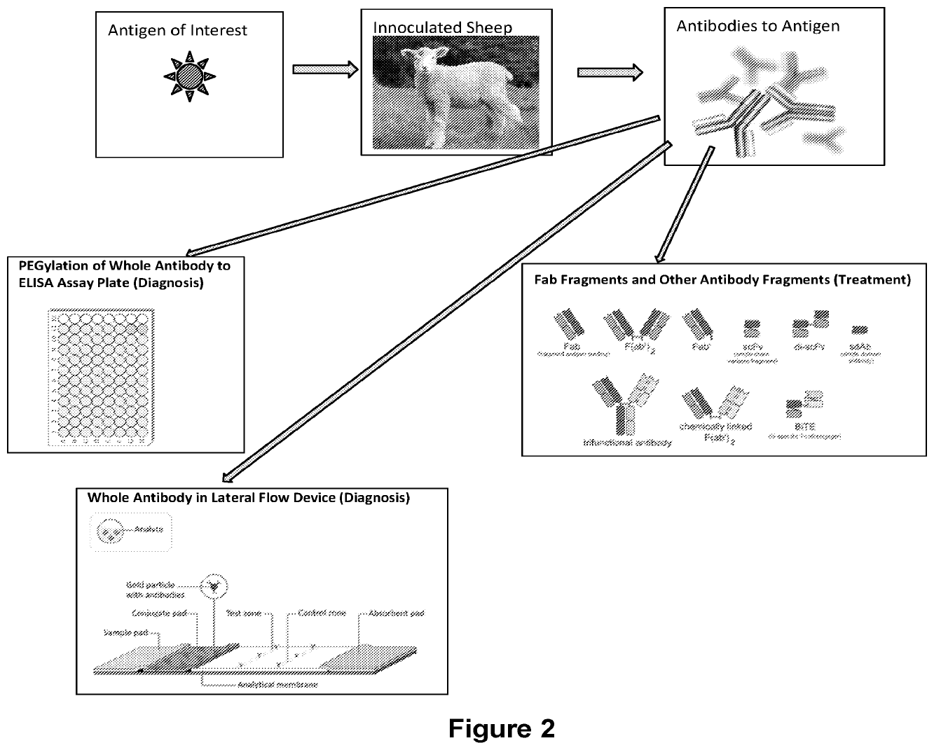

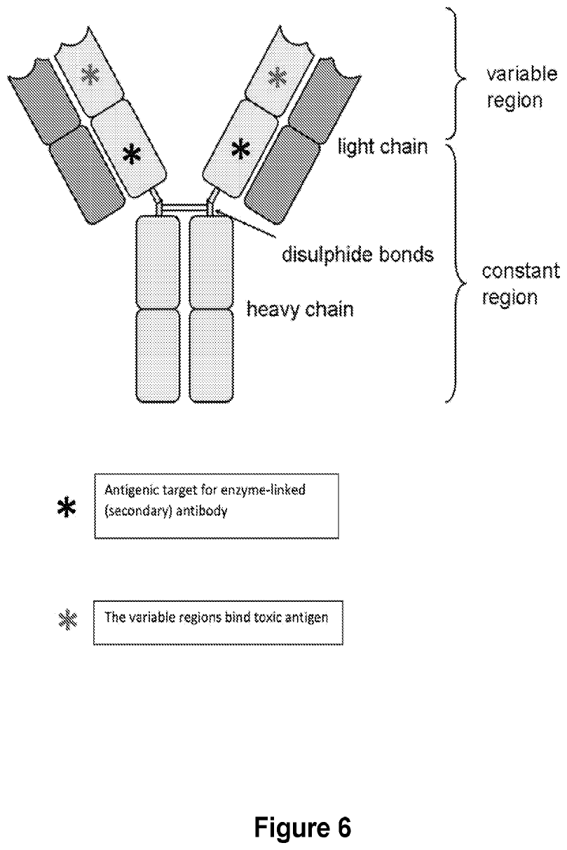

Antibodies for eventual use in Fab-DT are roughly Y-shaped, with two variable antigen-recognizing Fab (fragment antigen binding) portions and a constant portion comprising the stem (Fc) and adjacent proximal portions of the two Fab fragments. (See FIG. 2 and FIG. 6). The entire antibody, derived from a host animal, can be enzymatically cleaved to produce several types of antibody fragments, depending on the enzyme used (FIG. 2).

Enzymatically cleaved antibody fragments are preferable to whole antibodies within an RB-DT platform for two reasons. First, the Fc portion of whole antibodies binds to Fc receptors on immune cells, inducing inflammation. This inflammation is often detrimental, e.g., in the case of an inflammatory toxin or infection where there is already adequate or even pathological inflammation. Second, Fc and the proximal portions of the Fab fragments are constant regions, preserved within a given species, but varying amongst different species. These Fc portions from different species in particular may be immunogenic to a host of another species, especially upon repeated exposure. Host antibodies generated against the foreign Fc portion could result in negation of the intended therapeutic effect of the antibody product, pathological inflammation, or even anphylaxis.

It is understood that the RB-DT accomplished by Fab-DT could be approached via AT-DT or NA-DT within the limitations and specifications outlined above. So, when discussing Fab-DT, I am including other known (AT-DT and NA-DT), and currently unknown technologies, with the intrinsic capacity to serve at once as a diagnostic and a therapeutic, as does Fab-DT.

6. Sepsis: Current Art Vs. Fab-DT

Infectious organisms cause disease by releasing toxins and bacterial components which are toxic to the host, binding to specific host receptors and thereby disrupting normal physiologic processes [Hayk], [Ramachandran]. Additionally, bacteria may produce exoenzymes which catalyze reactions to digest tissue, disrupt immune responses and/or negatively affect homeostasis. Without these toxic substances, colonization, or in some cases even symbiosis would occur, but not disease. So, infection—as opposed to colonization—requires the presence of bacterial toxins. To that extent, infections may be considered poisonings with the specific bacterial toxin(s). Besides LPS (present on the surface of all Gram-negative bacteria) and Peptidoglycan (present on the surface of all Gram-positive bacteria), there are toxins unique to each clinically relevant infection that are characteristic to that infecting organism (see Table 1). Not all bacteria of a particular subspecies will possess the characteristic toxin. However, there is an association between certain species and specific toxins, and the toxins often serve as virulence factors, making severe infection more likely. Thus, the recognition and management of bacterial toxins can play a crucial role in the clinical outcome of septic patients. Each of these unique signature toxins produces a specific set of physiologic effects, together constituting a toxidrome. Once recognized, these toxidromes can be used to clinically drive antibiotic selection, supportive medical therapy, and surgical decision-making, similar to the way a toxic ingestion (e.g., acetaminophen overdose) might be approached. (See Table 1.)

So, an infection with toxin-producing organisms can be understood on some level as a poisoning: remove the toxin, stop the infection. However, in actual current practice, supportive therapy and even antibiotic selection are often generic, with little or no ability to tailor the medical response to the specific bacterial toxic situation unfolding in the patient.

In sepsis, time to administration of effective antibiotic and time to resolution of shock are the two most important determinants in survival controlled by the physician. Time is survival, whereas poor sepsis outcomes are observed when diagnosis and treatment are delayed [Judd], [Whiles], [Ferrer]. [Filbin], [Kumar], [Liu], [Pruinelli].

a. Sepsis Diagnosis

In the current art, antibiotics are begun empirically, having surmised a source of infection and assimilated host risk factors into a fuzzy logic of which organism is likely to be causing infection. The conclusions may or may not be correct. This will usually not be known with certainty until an infected tissue or fluid sample has been incubated in the lab to grow bacteria, which are then tested for various metabolic characteristics in order to identify their species and antibiotic susceptibilities. This generally requires three days, lagging well behind the clinical course. Any toxins produced by the infecting bacteria would still be unknown at that point. Treatment decisions would be made from knowledge of predicted characteristic toxins, expected course of a particular bacterial species and clinical signs in the patient—an inexact science at best. Additionally, cultures don't always grow infecting bacteria, so multiple samples are obtained over time and they are all incubated in the hopes that one or more will grow bacteria consistent with the clinical infection.

Fab-DT, is advantageous over the prior art in that allows for identification of an antibody panel and thereby identification of an infecting organism—and its armamentarium of toxins—in about 30-80 minutes. Further advantageous, Fab-DT provides a specific treatment regimen to combat the bacterial toxins driving the toxidrome.

b. Current Sepsis Treatment

In the current art, antibiotics are used to neutralize or destroy bacteria in the treatment of sepsis. However, emerging antibiotic resistance is a constant factor in this treatment modality. For instance, multi-drug resistant (“MDR”) “super-bugs” are becoming more frequent as catalogued by the CDC [Containment Strategy Guidelines|HAI|CDC]. Within an individual patient, this sometimes results in antibiotic failure midcourse, as pre-existing resistant bacteria are selected out by the administration of antibiotics, or may complicate treatment in the event of recurrence of the initial infection. Over the population, MDR bacteria are aggressive and difficult to treat, resulting in significant morbidity, mortality, and economic expenditure.

Antibiotics can also result in secondary infections. For example Clostridium difficile (C. diff) is the cause of “antibiotic-associated colitis”. C. diff is present in healthy people, but when antibiotics kill off infecting bacteria as well as healthy bacteria, the microbiome is significantly altered, and C. diff becomes triggered to produce two enterotoxins (“A” and “B”) which cause the colitis.

Antibiotics are also associated with direct toxic effects on the host human, such as renal injury, altered mental status, and drug interactions with other medicines. Further, antibiotics often lyse bacterial cells, causing the sudden release of bacterial toxins, sometimes producing acute worsening in the patient's condition, known as “Jarisch-Herxheimer (J-H) Reaction”. This sudden release of bacterial toxins carries clinical significance in many septic scenarios, including suspected pneumococcal meningitis. Administration of anti-inflammatory steroid 15 minutes prior to antibiotic dosing produces better clinical outcomes for patients with pneumococcal meningitis.

c. Alternative Strategies

Bacterial sepsis is a complex, dynamic cascade, driven by virulence factors and bacterial toxins. Virulence factors allow bacteria to escape host immune killing. Bacterial toxins interact with host receptors to cause injury and a dysregulated immune response, producing the phenotypic changes of sepsis, septic shock and multiorgan failure.

Antibiotics attempt to interrupt bacterial metabolism and structural integrity. Additionally, recent advances in treatment have focused on modification of dysregulated host responses, e.g., monoclonal antibodies to inflammatory mediators like Il-6, TNF-alpha, or IL-1. These latter therapeutics have met with variable success, as timing during the inflammatory cascade, comorbidities, and host genetic polymorphism all complicate the modulation of a single inflammatory mediator.

Therapeutics to neutralize bacterial toxins and other virulence factors would be expected to synergize with antibiotics, while avoiding the complicated issues of host variability outlined above.

d. Fab-DT Synergy With Antibiotics for Sepsis

With Fab-DT, previously prepared fragments of the same antibodies that identified the infecting bacterial toxins by ELISA, are administered intravenously, intramuscularly or subcutaneously to bind and neutralize these toxins in vivo. Fab fragments can be dosed 15-30 minutes before antibiotics, to avoid a Jarisch-Herxheimer (J-H) Reaction. In this way, Fab fragments have the advantage of working dissociate toxins off of host tissue, and serve as a sink for any toxin released by subsequent antibiotic-mediated destruction of bacteria.

While free toxin can interact with host receptors to cause illness, toxins bound by antibody fragments are not free to interact with host receptors. In essence, when all free toxin becomes bound, the host has reached a zero-toxin state, equivalent to a lack of infection clinically. After this stage, measures must be taken to ensure no further free toxin is produced and the source of that production eradicated or placed into a neutral state incapable of, or not inclined to engage in, further toxin production. Fab fragments, including those of the Fab-DT model disclosed herein are advantageous and antibiotics can synergize in reaching the zero-toxin state.

Bacterial toxins can be broken into three broad reservoirs or sources during an infection: (1) toxin already released from bacteria, free to interact with host receptors; (2) toxin already produced but still residing inside bacteria, which can be unintentionally liberated by bacteria-lysing antibiotics which interact with host receptors if a Fab fragment sink is not in place; (3) future production of toxin by new or unkilled bacteria during a current infection. Only Fab fragments can address the post-translational, free circulating bacterial toxin. We know from the pharmacodynamics and clinical observations of DigiFab and CroFab (therapeutic Fab fragments for Digoxin overdose and poisonous snake bites from Crotalidae) that significant clinical improvement can remarkably be expected within 15-30 minutes. For the second reservoir, i.e., preformed toxin inside of bacteria, Fab fragments and antibiotics can be synergistic. Antibiotics stop the bacterial cell from functioning and this may compromise integrity or fully kill the bacteria. Either way, pre-formed toxins are liberated into the host circulation.

The current disclosure is advantageous in that in a Fab fragment antidote, selected by Fab-DT, is already present in the host circulation, and the liberated toxin will bind to the Fab fragments, rather than to host cell receptors. Antibiotics also help prevent the formation of the third reservoir, namely future toxin production, by inhibiting ribosomal translation directly, or by killing the bacteria that would have later made toxin. Fab fragments help restore host integrity by ridding the host of bacterial toxins, making the host less susceptible to further bacterial infection and immune evasion during the current illness. Thus, Fab fragments and antibiotics also synergize in regards to the third reservoir of bacterial toxin.

Finally, by directly absorbing toxin from the three reservoirs, Fab-DT hastens arrival to the zero-toxin state, promotes earlier host healing, and should thereby shorten the required antibiotic course. This would in turn limit the emergence of antibiotic resistance, prevent secondary infections via minimizing microbiome disruption, and also reduce direct toxic effects on the host by antibiotics (e.g., acute kidney injury).

e. Fab-DT for Nonbacterial Infections

The same principles of proposed Fab-DT action in bacterial sepsis and synergy with antibiotics also apply to infections from nonbacterial microbes, their toxins, and synergy with antimicrobial pharmaceuticals directed at them. These nonbacterial infections include archaea, and also eukaryotes like fungi and protozoa.

Viruses are also amenable to Fab-DT. We have seen the role of antibodies in both diagnosing and treating viruses during the recent COVID-19 pandemic. Fab-DT is advantageous in that it allows for advancement of this approach, whereby patients are given a diagnosis of a particular COVID variant. The Fab-DT allows for inclusion of as many antibody preparations for infusion as there are significantly different (immunogenically) SARS-CoV-2 variants. The antibody Fab fragment is further advantageous in that the therapy would always match well to the identified variant. Highly effective antibody fragment binding, e.g., in the case of SARS-CoV-2, to S-glycoprotein, prevents host cell entry. Additionally, utilizing a Fab fragment might allow utility later in infection than the recently used monoclonal preparations, since the latter contain Fc portions. Without those Fc portions, Fab fragments avoid increasing Fc-driven inflammation later in disease when some patients may already be headed toward pathological inflammation, i.e., cytokine storm.

There should be expected synergy between Fab-DT and antivirals, such as nucleotide analogs (Remdesivir for SARS-Cov-2, Famcyclovir for Herpesviruses, etc.) and protease inhibitors (Paxiovid for SARS-CoV-2, Simeprevir for Hepatitis C, Darunavir for HIV, etc.).

Fab-DT is limited by viral infections in one unique way: Fab fragments do not have ready access to the intracellular space.

7. Toxic Ingestions & Exposures: Current Art vs. RB-DT

Toxic Ingestions pose numerous unique challenges. For example, there are over 20 pharmaceuticals which can kill a 10-kg toddler in 1 or 2 doses [Koren], [Middlesex]. These include Camphor, Salicylates (especially Oil of Wintergreen), Sympatholytics (e.g., Clonidine patch), Calcium Channel Blockers (e.g., Verapamil), Narcotics, and Sulfonylureas. Treatment of toxic ingestions of many drugs, including the most lethal listed above, are limited as they do not have a specific antidote, and for many drugs, serum levels are not readily available in real time. Decontamination by inducing emesis or by charcoal has fallen out of favor due to difficult administration, the risk of aspiration, and because most victims present outside the clinical time window of utility for decontamination. Standard care is to observe the patient using vital signs and serial clinical exam to determine if there is a poisonous effect manifesting in the child. Only then is supportive therapy administered, and consideration given to specific antidotes, if available. There is a need within the art to more rapidly and specifically identify whether a dangerous toxin has been taken, if so in what amount, and to then treat only dangerous poisonings specifically and immediately.

Many overdoses occur intentionally in adolescents and adults. These patients may be intoxicated and/or suicidal at the time of presentation, and so reporting of overdose type, quantity and timing is unreliable. Alcohol or co-ingestants are common with presentation often delayed. The clinical approach is similar to that with toddlers, except that serum levels for various toxic alcohols, osmolality, acetaminophen and salicylate are sent, and often repeated at 4 hours. The clinician must search for an unknown toxin from a mental panel of suspects, and gradually refine consideration while providing medical support and specific treatments for the most likely or most dangerous suspects. There is a need within the art for more rapid and more specific diagnosis and treatment.

The current disclosure is ideal for improvement by Fab-DT, which could identify the unknown toxin(s) within as little as 30 minutes, and provide a specific, rapidly acting antidote. The Fab-DT reduces or eliminates the need for dialysis, particularly in those patients that present early after ingestion. There would be expected greatly decreased risk to the patient, and significantly reduced cost of care. Expectations of clinical course and outcomes are derived from a wealth of experience with the isolated stand-alone (not part of a platform) antidotes DigiFab and CroFab.

Common adolescent and adult ingestions which lead to the most fatalities include [Miller]: Benzodiazepenes, narcotics, barbiturates, antidepressants, antidiabetics, alcohol (ethanol; and less commonly ethylene glycol or methanol), and also anti-hypertensives, acetaminophen, and salicylates. All of these drugs can precipitate the need for emergency intubation and mechanical ventilation. Most of these drugs can necessitate the need for emergency dialysis, and for critical care in the ICU to provide adequate medical support.

Of these common dangerous adolescent and adult ingestions, only narcotics and the alcohols have a specific antidote which can be used in overdose emergencies: Narcan is an excellent opioid reversal agent, but has a short half-life; Fomepizole inhibits alcohol dehydrogenase and, when given very soon after ingestion, can prevent the metabolism of toxic alcohols to their toxic metabolites. (Flumazenil can reverse benzodiazepine binding at GABA receptors, but can precipitate refractory seizures in patients with chronic benzodiazepine use, and so is rarely used clinically.) Every one of these drugs, with the exception of the alcohols (small molecular profile, low antigenicity), is amenable to specific Fab fragment development, and could be addressed by Fab-DT. Notably, Fab fragments circulate for several days, so do not have the issues posed by short-acting pharmaceutical antidotes (e.g., Narcan or Flumazenil).

8. Bioterrorism Threat Response: Current Art vs. RB-DT

Despite the known existence and extensive description of many potential bioterrorism agents (above, and Tables 3 & 4), the availability of treatment modalities directed against such agents and toxins poses significant and unique challenges. Most toxins and infectious agents amenable to bioterrorism are genetically, evolutionarily, and structurally diverse, thereby necessitating therapeutics tailored against each agent or toxin. In addition, many of these agents and toxins can induce morbidity and even mortality within a matter of hours before a definitive etiologic diagnosis. Further, the earliest clinical manifestations of many select agents and toxins are indistinguishable from each other and thus a combinatorial treatment approach is required without complete knowledge of the toxin or infectious agent being treated. There exists in the art the need for the ability to rapidly identify toxins and infectious agents following possible exposure and provide effective treatment modalities.

In the current art, surveillance for a bioterrorism event utilizes specifically trained canines in airports, a pattern of purchases by a nefarious agent, intercepted communications, and the possible pre-strike case due to accidental contamination of the would-be terrorist. These methods are all very limited in the ability to detect/predict and prevent a bioterrorism event. In most practice scenarios, a mock bioterrorism event is detected by the symptoms in the first victims, and secondary signs like a gas or liquid sprayed over a population, and particular odors or tastes or reports of mucosal membrane irritation. Such detection would be very late and would very likely be followed soon thereafter by an overwhelming number of similar victims. So, there exists within the art the need to detect bioterrorism agents at very low levels before symptoms develop in victims, and hopefully even before would-be victims are exposed.

Likewise, in the current art there are very few specific antidotes for the numerous biological and chemical bioterrorism agents the CDC considers most likely to be used. (See Tables 3 and 4 for the 29 biological toxins and 13 classes of chemical toxins of concern). Under appropriate situations, the military supplies its soldiers with a nerve gas antidote kit containing acetylcholine and 2-PAM in a cartridge, which fits into an autoinjector (similar to Epi-Pen), and is extremely effective. The almost immediate onset of symptoms and rapid progression to paralysis and death require such an immediate response self-administered by each victim.

For other agents, there is no ready antidote currently. The specific toxin or agent used in a bioterrorism attack must be surmised clinically as detailed above, and then confirmed in a reference lab, likely to be hours away, by assay techniques that often require several hours to days. This is simply inadequate to address a mass event in the military arena, or in the public space.

Fab-DT is advantageous in that it can provide the solution to a mass bioterrorism event. Consider the example of a military exposure. For instance, lyophilized powder of Fab antibody fragments for reconstitution directed against agents/toxins of concern would be produced beforehand. In one embodiment, a cartridge to fit the same autoinjector as used for nerve gas contains lyophilized powder and reconstitution fluid. After rapid manual reconstitution (by any number of already available methods), the Fab cartridge, could be used in conjunction with an injection device, such as an Epi-pen to self-administer the Fab antidote.

Fab-DT is further advantageous as it allows for detection of a bioterrorism exposure from continuous environmental surveillance of air, soil, etc. and the presence of exposure could be confirmed using bodily fluids. The Fab-DT disclosed herein provides the additional advantages of being easy to administer and the assays easy to perform.

A very similar scenario can be imagined of Fab-DT diagnosing and treating the public in the event of a mass bioterrorism event. The emergency departments would serve as the place of diagnosis and initial treatment, in conjunction with point-of-care testing or testing in the hospital laboratory on site. Fab antidotes would be stockpiled at either military bases or Poison Centers, and distributed to Emergency Departments at the time of recognition of the event. Emergency Department personnel would administer antidote rapidly via injection.

### 9. Novel Assay Systems for Accomplishing Fab-DT

Disclosed herein are two novel immunoassays for use in Fab-DT. The Multiplex Lateral Flow Device (MLFD) is technically advantageous as it is very straightforward, can be positioned in most environments, and is technically simple to perform. MLFD is ideal for bioterrorism surveillance/detection and diagnosis and can be used for Fab-DT in the context of sepsis or toxicologic poisonings.

The second novel immunoassay is a Modified Competitive ELISA” (MC-ELISA). MC-ELISA uses a standard anti-Light Chain (anti-LC) secondary antibody in the assay, affording numerous benefits described in the second section below.

a. Multiplex Lateral Flow Device (MLFD)

As used herein, the term “Multi-Lateral Flow Device” (MLFD) or “Lateral Flow Device” refers to a single well that can be used to receive solubilized samples of interest and distribute sample to various Lateral Flow Devices (LFD) arranged in parallel as part of a Multi-Lateral Flow Device (MLFD). The lateral flow device can be designed as a single chamber or multi chamber in a single test. A multi-channel device allows for the detection of multiple antigens in a single read.

Multiple antibodies attached to different colors and directed at different antigens can be employed within the same test strip in an array format and/or through parallel alignment of multiple assay devices with a shared sampling well. This latter option of parallel alignment is a novel proposal and is henceforth referred to as “Multiplex Lateral Flow Device” (MLFD). Other potential labels for use in MLFD's include fluorescence [Li], [Song], [Venkartaman], paramagnetism [Liu], [Wang], enzymatic reaction [Mirasoli], [Maiolini], and colored carbon nanoparticles [Qiu]. Likewise, nucleic acids can be employed to bind and amplify certain sequences of interest and would have application to NA-DT.

b. Surveillance: Beyond Diagnosis and Treatment

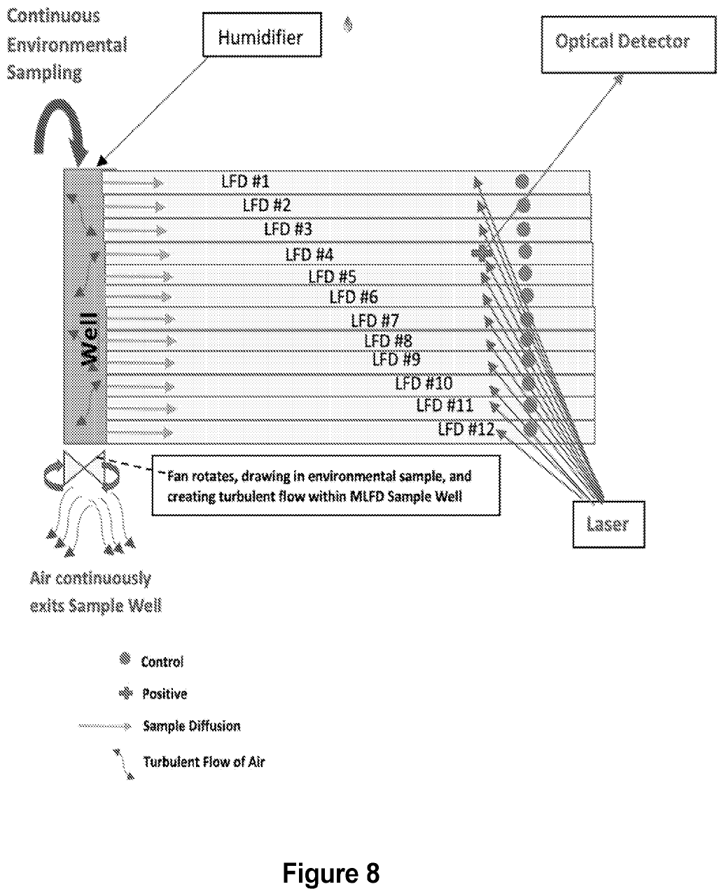

In a further embodiment, a humidified air sampling component is added to an assay instrument. For example, a small fan attached to the sample well of the MLFD described above, allows for continuous aerosol and airborne sampling, enabling detection before symptoms or even before a toxic level of exposure (See FIG. 8). This embodiment has application in numerous real-world settings, including but not limited to the military for use by troops to detect threat/exposure before symptoms and hopefully before biologically significant exposure; in high-risk areas like subway systems, convention centers, government buildings; monitoring of water reservoirs; in laboratories working on bioterrorism and chemical terrorism agents to detect incomplete sequestration/accidental leak very early and at subclinical levels to prevent larger exposures.

c. Modified Competitive ELISA (MC-ELISA)

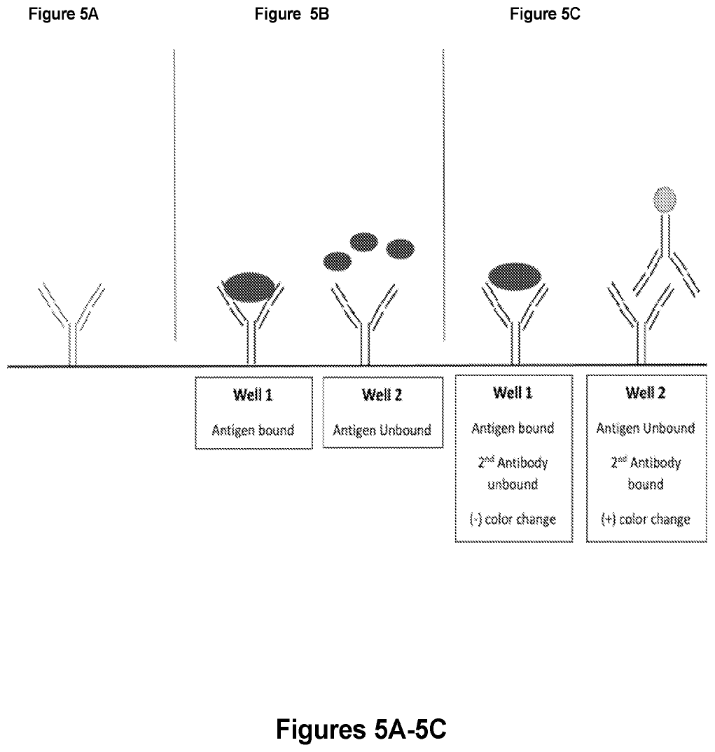

Modified Competitive ELISA (MC-ELISA) is a novel approach to ELISA that identifies antigens present in a body fluid by utilizing an array of antigen-specific primary antibodies, each chemically adherent (e.g., PEGylated) to an ELISA plate well, and a standard secondary enzyme-linked antibody specific for the Light Chain Constant (LC) portion of the primary antibodies (see FIG. 5). The MC-ELISA works on the principle that if the antigens in question are present in the sample, each antigen will bind specifically to a single monoclonal primary antibody adherent to an individual ELISA well. When the standard anti-LC secondary antibody is added to all the wells, it will only bind those primary antibodies for which antigen is not bound or is weakly bound, allowing the secondary antibody access to the LC area. Thus, photometric signal from the enzyme-linked secondary antibody will indicate an absence or relative paucity of the antigen in question for each well. A greater signal means less antigen is present; the inverse is also true.

More specifically, as used herein, the term “modified competitive bioassay” (MC-ELISA) refers to an ELISA that is modified for rapid detection of an infectious agent or toxin. Clinically relevant infections require toxins to be efficacious, identification of the characteristic surface antigens and antigens of intrinsic toxins by the use of antigen-specific antibody Fab fragments, as disclosed herein, permits identification of the infecting organism or any variants of the infecting organism. This allows for rapid identification of infectious agents and toxsin, including for example bacterial infections, viral infections, and plant- and animal-derived toxins (accidental, intentional, and bioterrorism contexts). Rapid identification (minutes vs. days for traditional culture and typing) of an organism and its particular toxic properties, such as possession or absence of specific toxins allows appropriate diagnosis and medical decision-making. Likewise, rapid identification of poisonings (e.g., ricin, strychnine) is crucial to effective treatment and survival, especially in a mass event.

In a preferred embodiment, a unique antibody, monoclonal antibody, monoclonal antibody, or fragments thereof, including Fab, (Fab′2), or fragments thereof (Fv, Fab, F(ab′)2, Fab′, dsFv, (dsFv)2, scFv, sc(Fv)2, diabodies) with specificity for a single toxin is adhered to each well of an assay plate. In an alternative embodiment In one embodiment the plate can be customized with multiple antibodies or fragments thereof and multiple infectious agents or toxins detected in a sample. Thus, each plate represents a wide array of toxin antigens and/or infectious agent antigens. In another embodiment a plate can contain a single antibody, monoclonal antibody, or fragments thereof, including Fab, (Fab′2), or fragments thereof (Fv, Fab, F(ab′)2, Fab′, dsFv, (dsFv)2, scFv, sc(Fv)2, diabodies) and multiple samples analyzed simultaneously. In yet another embodiment the plate can allow for detection of multiple toxins or infectious agents from multiple samples. In still yet another embodiment the antibody or fragment thereof is adhered to non-plate support such as a lateral flow device, capillary tube, or membrane. The sample is then added to the wells. In one embodiment the sample is peripheral blood, sera, plasma, ascites, urine, cerebrospinal fluid (CSF), sputum, saliva, bone marrow, synovial fluid, aqueous humor, amniotic fluid, cerumen, broncheoalveolar lavage fluid, semen, prostatic fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, purulent exudate, lavage fluids from sinus cavities, bronchopulmonary aspirates, umbilical cord blood, tissue culture medium, tissue extracts such as homogenized tissue, tumor tissue, cellular extracts, or combinations thereof. In additional embodiments plant material, including portions that adsorb or absorb toxins, environmental samples, mechanically or biologically processed or nonprocessed, e.g., water supply, and/or gas or vapor condensed to liquid via cooling and or pressurization can be used in as the starting sample.

In a preferred embodiment of the assay system, antigens compete for binding of the constant chain of the primary antibody adhered to the plate or support. The more specifically the antibody recognizes the antigen, the more avidly it will bind to the antigen, and the less enzyme-linked secondary antibody will bind. Therefore, the amount of secondary antibody bound is inversely related to amount of sample-based antigen bound to the antibody. The result will propagate in the luminescent produced by the enzyme-linked secondary antibody. More specifically, the less toxin or infectious agent in the sample, the more binding sites remain available for a secondary antibody binding to the primary antibody bound to the well, which is reflected as increased luminescent in the well for a given antibody. Moreover, the presence of increased levels of a toxin or infectious agent in a sample is manifested as fewer binding sites available to the secondary antibody, and the less luminescence or quantifiable signal that will be produced and/or quantified.

Modified competitive bioassays are technically advantageous when compared to current methods for identification of infectious agents, which requires identification via bacterial culture of a biological sample. Culture of biological samples requires growing and propagating an organism in culture medium, identifying organisms, and testing metabolic assays for specific subset identification, and then determining antibiotic susceptibility by growth or recession of colonies. This method is cumbersome, slow, and yields no specific antidotes, but rather broad-spectrum medicines in the form of antibiotics. In contrast the modified competitive bioassay of described herein allows for rapid determination of the toxin or infectious agent, increased specificity, and insight into the treatment modality that is appropriate for the toxin or infectious agent identified.

The modified competitive ELISA (MC-ELISA) is advantageous over a traditional competitive ELISA as the latter is comprised of a unique monoclonal antibody PEGylated to a well. Samples are added and the antigen of interest bound to the antibody in a well. Then enzyme-linked versions of each antigen must be added to the appropriate well to compete with the sample antigens for binding to the PEGylated antibodies. Failing to add the correct enzyme-linked antigen to a well with the proper antibody produces a false positive signal for the antigen in question from the sample. MC-ELISA avoids this issue by having a single, standard, enzyme-linked anti-HC antibody added to every ELISA well of the test plate after sample antigens have been allowed to incubate with the primary antibody in each well.

Additionally, with traditional Competitive ELISA, antigenic variants in a given sample, with an antigenic epitope recognized by the fixated antibody, will result in in decreased binding to the PEGylated antibody; interpreted as a lower concentration of antigen, without recognition of a variant. In contrast, with a two-step MC-ELISA, a low binding-affinity enzyme-linked secondary antibody can be added to the all of the wells to screen for antigen binding, followed by a higher-affinity enzyme-linked secondary antibody, which should displace variants of the antigenic epitope, and will enable calculation of a binding affinity curve. This simultaneously identifies variants and determines how effective a given antibody might be at binding the variant antigen in vivo as a clinical treatment.

By keeping the specificity of the enzyme-linked agent constant (affinity for HC portion of PEGylated antibody), MC-ELISA decreases operator error and enhance ease of production and laboratory use. By varying the coefficient of binding while keeping the anti-HC specificity the same, a two-step MC-ELISA should be able to identify variants Bad in the Fab-DT paradigm, calculate the clinical utility of available Fab antidotes.

The modified competitive bioassay of the current disclosure is also advantageous over assays in the art as it allows for identification of infectious pathogens and toxins in near real time. Currently antibodies are used to diagnose infections and the presence of conditions including HIV or Hepatitis, or Lyme disease, for example. However, characterization of these pathogens and their variants require techniques that are time-consuming requiring labor-intensive techniques and hours or days to generate a specific toxin or infectious agent characterization. The assay system of the current disclosure can utilize a wide-range of sample types and antibody panels to identify the presence of a toxin or infectious agent resulting from bacteria and their active toxins, bioterrorism agents, or viruses. Furthermore, once identified, the assay system disclosed herein identifies the specific precision treatment required to address the infections agent or toxin. This eliminates the need to guess the origin of an infective agent and required treatment.

The MC-ELISA also produces a stronger visualized and/or quantifiable signal when the sample contains a low quantity of antigens. This results in the MC-ELISA being a very sensitive assay, even for samples with a small number of antigens, allowing for early and accurate detection of infectious and toxicologic agents. The assay is further advantageous over traditional Competitive ELISAs in that the binding an antibody to the plate of the assay system or lateral flow device allows the entire antigen of interest to be available for antigenic recognition. In contrast, in the traditional Competitive ELISA, binding the antigen to the assay plate could cause antigenic alteration or masking. Further, in the MC-ELISA only a single unique secondary enzyme-linked antibody is required since it only needs to recognize one antigen: the heave chain constant region of the affixed primary antibody. This reduces potential error in the application of the secondary enzyme-linked antibody, since only one standard antibody is required, rather than matching primary and secondary antibodies with same antigenic affinities.

The MC-ELISA is a novel approach to ELISA technology in that it allows for identification of antigens present in a sample, utilizing an array of antigen-specific primary antibodies, each adfixed to an assay plate well or lateral flow system. A standard secondary enzyme-linked antibody specific for the Heavy Chain Constant (HC) portion of the primary antibodies can then be used in the assay well. If the antigens in question are present in the sample, each antigen will bind specifically to a single monoclonal primary antibody adfixed to an individual ELISA well. When the standard anti-HC secondary antibody is added to all the wells, it binds only primary antibodies for which antigen is not bound or is weakly bound, allowing the secondary antibody access to the HC area. Thus, a visualized and/or quantifiable enzyme-linked secondary antibody will indicate an absence or relative paucity of the antigen in question for each well. A greater signal means less antigen is present and a reduced or absent signal means more antigen is present in the sample.

d. Mc-ELISA Technical Advantages Over Sandwich ELISA

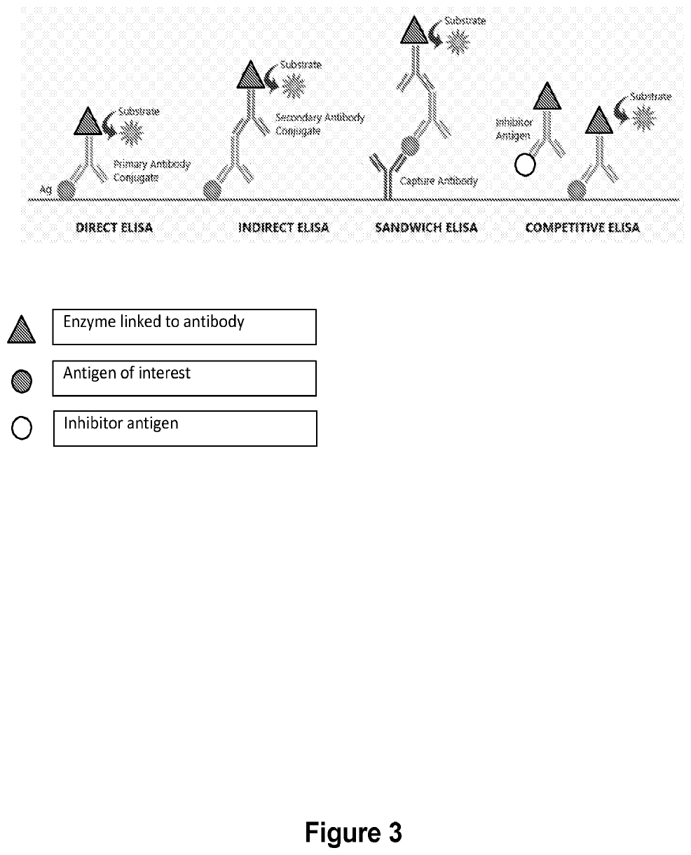

Sandwich ELISA requires three antibodies: one chemically adherent primary antibody in each cell, and one soluble secondary antibody, each specific for a different epitope of the antigen in question (“X1” and “X2” respectively); and an additional tertiary enzyme-linked antibody specific for the Heavy Chain (HC) constant region of the second antibody. (See FIG. 3.) This assay requires the appropriate placing of secondary anti-“X₂” antibody into the same well as primary anti-“X₁” antibody. This matching of appropriate antibodies in each well for each antigen in question increases the likelihood of technical error.

MC-ELISA, in contrast, allows for easier production and reduces potential error by standardizing a single secondary enzyme-linked antibody. A standardized secondary antibody also avoids the failure of sandwich ELISA to detect variants in the case of infectious agents which have altered the epitope for the secondary antibody. By targeting the LC portion of the primary antibody (which is unchanging), MC-ELISA only requires unique recognition of one epitope (“X1”), instead of two (“X1” and “X2”).

Sandwich ELISA are also prone to antigenic drift or shift (natural or engineered) in infectious antigens may render, e.g., the primary antibody avidly binding the antigen, but the secondary antibody failing to bind well or at all to the antigen. The consequent false negative result would, in the context of Fab-DT, fail to identify an important body fluid antigen and thereby fail to identify an important potential treatment.

Thus, Sandwich ELISA has difficulty identifying antigenic variants. While multiple primary antibodies can be fixed to a plate, one monoclonal antibody preparation to each well, to bind a variety of antigenic variants of a particular target, sandwich ELISA would also require a similar array of secondary enzyme-linked antibodies, each with an affinity for a different variant. The combined numbers of primary and secondary antibodies needed would become multiplicative. This is technically confounding and inefficient, and as outlined above, likely to result in false negatives. Modified Competitive ELISA (MC-ELISA) solves these technical inefficiencies and inaccuracy in a simple two-step process, and lends itself to the detection of variants, by keeping the secondary antibody standard.

In Fab-DT, recognition of an antigen by a specific primary antibody in vitro drives therapy with the Fab fragment version of that primary antibody in vivo. Optimizing detection of antigen is essential, as is accurate assessment of the binding coefficient. Increased bound antigen prevents binding by the secondary enzyme-linked antibody and therefore results in a fainter photometric signal. This fainter signal may indicate increased concentration of the antigen of interest, or increased coefficient of binding by a variant of the antigen of interest. This can be further elucidated by a 3-step MC-ELISA, in which initial detection occurs as outlined above, and the assay is then repeated with a new secondary enzyme-linked anti-LC antibody possessing a stronger coefficient of binding than the previous one. Thus, a 3-step MC-ELISA can be easily performed using at first a weak anti-LC enzyme-linked antibody, followed up by a strongly binding anti-LC enzyme-linked antibody. Since coefficients of binding between primary antibody and common antigens would be known a priori, changes in photometric signal between the first (low affinity) and second (high affinity) application of secondary anti-LC antibody in the 3-step MC-ELISA should follow a standard curve. If there is a steeper change than expected between the two steps, this would indicate the coefficient of binding may be different than that of the previously known antigen, identifying a variant.

Additionally, the greater the coefficient of binding, the greater clinical utility/treatment effectiveness could be expected from the Fab version of the primary antibody in vivo. The opposite is also true. This clinical utility cannot be easily elucidated by standard sandwich ELISA.

Thus, as disclosed herein, the MC-ELISA, overcomes these limitations by production of a single standardized secondary enzyme-linked antibody, thereby reducing potential error by standardizing the secondary antibody. A standardized secondary antibody also avoids the failure of sandwich ELISA to detect variants in the case of infectious agents which have altered the epitope for the secondary antibody. By targeting the HC portion of the primary antibody, MC-ELISA only requires unique recognition of one epitope, instead of two.

e. Technical Advantages Over Traditional Competitive ELISA

To test for the presence of various antigens in a sample body fluid, Competitive ELISA wells each contain a unique antigen of interest chemically coated to the well. The entire plate of up 96 wells creates a panel of antigens of interest. The unknown sample fluid is separately incubated with a unique solution of enzyme-linked antibody against each particular antigen of interest. Each mixture is then added to the corresponding well for a given antigen of interest. If the antigen was present in the sample fluid, then less unbound antibody will be available to bind the coated antigen in the well. (See FIG. 3).

After a wash of the wells, any antibody bound to antigen of interest from the body fluid will not have become bound to chemically adherent antigen in the well, and the soluble antibody-antigen complexes will be washed away; only antibody bound to chemically-fixed antigen will remain. Thus, chemically-fixed antigen in each well competes with any antigen in the sample fluid for binding to enzyme-linked antibody. Both the enzyme-linked antibody and the chemically fixed antigen in the wells need to be matched property to the same well, introducing possible operator error. In the final step, substrate is added to interact with any enzyme-linked antibody remaining in the well. Traditional Competitive ELISA relies on chemically coating each well with antigen of interest. In contrast, MC-ELISA relies on competition between antigen and standardized enzyme-linked anti-LC antibody for binding to chemically coated primary antibody. By removing one variable (standardizing the secondary antibody), potential for operator error is reduced.

Additionally, with traditional Competitive ELISA, if there is a variant of the antigen in question in the body fluid, and if that variant involves the antigenic epitope recognized by the enzyme-linked antibody, then binding to the chemically fixed antigen in the well will be decreased. This would be interpreted as a lower concentration of sample fluid antigen, without recognition of a variant. In contrast, with a 3-step MC-ELISA, a low binding-affinity enzyme-linked secondary antibody can be added to the all of the wells to screen for antigen binding, followed by a higher-affinity enzyme-linked secondary antibody, which should displace variants of the antigenic epitope away from the primary antibody, and will enable calculation of a binding affinity curve for the antigen. This simultaneously identifies variants and determines how effective a given antibody might be at binding the variant antigen in vivo as a clinical treatment.

By keeping the specificity of the enzyme-linked agent constant (affinity for LC portion of PEGylated antibody), MC-ELISA should decrease operator error and enhance ease of production and laboratory use. By varying the coefficient of binding while keeping the anti-HC specificity the same, a 3-step MC-ELISA is able to identify variants and in the Fab-DT paradigm, calculate the clinical utility of available Fab antidotes.

Both the 2-step MC-ELISA and the 3-step MC-ELISA can be further simplified by adding the secondary enzyme-linked anti-LC antibody to every well at the factory, producing a 1-step MC-ELISA. This allows a single step to be performed with addition of sample fluid to each well and then washed. If antigen is present in the sample fluid, it will displace the enzyme-linked anti-LC antibody, decreasing the photometric signal. This allows the same results as the 2-step MC-ELISA described above. Using sequential wells with enzyme-linked antibodies of increasing anti-LC affinity allows the same functionality as the 3-step MC-ELISA described above. The 1-step MC-ELISA has greater utility at bedside or in the field, since is technically much simpler for the operator.

f. MC-ELISA Conclusions

In many cases where there is still partial affinity of a variant antigen for the primary antibody, MC-ELISA enables rapid and technically simple detection and quantification of newly arising variants (viruses or toxins), which is critical during pandemics like COVID or Influenza, or epidemics like Ebola, or chemically modified bioterrorism agents. Additionally, MC-ELISA offers the ability to calculate the clinical utility of already prepared antidotes (the Fab fragments of the primary monoclonal antibody in a given well) in the Fab-DT paradigm. These are significant gains over other existing ELISA strategies.

10. Combining MLFD with MC-ELISA: A Third Novel Assay for Accomplishing Fab-DT

Regarding LFA's, direct testing is used for larger analytes with multiple antigenic sites, whereas a competitive displacement assay is used for small molecules with single antigenic determinants [Workman], [Butler]. This competitive displacement assay inside LFAs could employ the novel methods of MC-ELISA to accomplish detection of small analytes. When several such LFAs are arranged in parallel in and MLFD format, utilization of MC-ELISA principles might be ideal, especially in the case of toxic materials, so they would not have to be handled in the production of the assay; i.e., the competitive assay would be based on displacement by secondary antibody, rather than displacement by toxic antigen. This MC-MLFD is a third novel assay proposal, as a combination of the first two proposals above (MLFD and MC-ELISA).

### REFERENCES

Singer M, et al. JAMA. 2016; 315(8):801-810. doi:10.1001/jama.2016.0287)
Martin G S. Sepsis, severe sepsis and septic shock: Changes in incidence, pathogens and outcomes. Expert Rev Anti Infect Ther 2012; 10:701-706.
Angus D C, Linde-Zwirble W T, Lidicker J, et al. Epidemiology of severe sepsis in the United States: Analysis of incidence, outcome, and associated costs of care. Crit Care Med 2001; 29:1303-1310.
Liu V, Escobar G J, Greene J D, et al. Hospital deaths in patients with sepsis from 2 independent cohorts. JAMA 2014; 312:90-92.
Torio C M, Moore B J. National Inpatient Hospital Costs: The Most Expensive Conditions by Payer. 2013. Available at: https://www.hcup-us.ahrq.gov/reports/statbriefs/sb204-Most-Expensive-Hospital-Conditions.jsp. Accessed Sep. 7, 2017.
Rudd K E, et al., “Global, regional, and national sepsis incidence and mortality, 1990-2017: analysis for the Global Burden of Disease Study.” Lancet. 2020; 395(10219):200-11.
Hayk M, “Sepsis: mechanisms of bacterial injury to the patient.” Scandinavian J Trauma, Resuscitation and Emerg Med, February 2019; 27(19).
Gotts J E, Matthay M A. Sepsis: pathophysiology and clinical management. British Medical Journal 2016.f sepsis
Hajj J, et al., The “Centrality of Sepsis”: A Review on Incidence, Mortality, and Cost of Care, Healthcare. 2018 September; 6(3): 90. doi: 10.3390/healthcare6030090
Gummin D D, et al. “2020 Annual Report of the American Association of Poison Control Centers' National Poison Data System (NPDS): 38th Annual Report.” Clin Toxicol (Phila). 2021 December; 59(12):1282-1501. doi: 10.1080/15563650.2021.1989785.
Mowry J, Spyker D A, Cantilena Jr L R, Bailey J E. Ford M. 2012 annual report of the American Association of Poison Control Centers' National Poison Data System (NPDS): 30th annual report. Clin Toxicol [online] 2013 [cited 2014 Jun. 3]; 51: 949-1229. Available from URL: https://aapcc.s3.amazonaws.com/pdfs/annual_reports/2012_NPDS_Annual_Report.pdf
CDC, National Center for Injury Prevention and Control. WISQARS injury mortality reports, 1999-2010 [online]. 2013 Feb. 19. [cited 2013 Nov. 18]. Available from URL: http://webappa.cdc.gov/sasweb/ncipc/mortrate10_us.html.
National Capital Poison Center-Poison Statistics National Data 2020|Poison Control
Botulism|Botulism|CDC
What is Ricin Poisoning?|Soligenix
Judd W R, et al. “Clinical and economic impact of a quality improvement initiative to enhance early recognition and treatment of sepsis”. Ann Pharmacother 2014; 48:1269-1275. doi: https://doi.org/10.1177/1060028014541792
Whiles B B, et al. “Increased time to initial antimicrobial administration is associated with progression to septic shock in severe sepsis patients.” Crit Care Med 2017; 45:623-629. doi: https://doi.org/10.1097/CCM.0000000000002262
Ferrer R, et al. “Empiric antibiotic treatment reduces mortality in severe sepsis and septic shock from the first hour: Results from a guideline-based performance improvement program.” Crit Care Med 2014; 42:1749-1755. doi: https://doi.org/10.1097/CCM.0000000000000330
Filbin M R, et al. “Sepsis visits and antibiotic utilization in U.S. emergency departments*.” Crit Care Med 2014; 42:528-535. DOI: 10.1097/CCM.0000000000000037
Kumar A, et al. “Duration of hypotension before initiation of effective antimicrobial therapy is the critical determinant of survival in human septic shock.” Crit Care Med 2006; 34:1589-1596. doi: https://doi.org/10.1097/01.CCM.0000217961.75225.E9
Liu V X, et al. The timing of early antibiotics and hospital mortality in sepsis. Am J Respir Crit Care Med 2017; 196:856-863. doi: https://doi.org/10.1164/rccm.201609-18480C
Pruinelli L, et al. “Delay within the 3-hour surviving sepsis campaign guideline on mortality for patients with severe sepsis and septic shock.” Crit Care Med 2018; 46:500-505
Ramachandran G, “Gram-positive and Gram-negative bacterial toxins in sepsis.” Virulence, January 2014; 5(1): 213-218. https://www.doi.10.4161/viru.27024 Containment Strategy Guidelines|HAI|CDC
Adalja A A, “Clinical Management of Potential Bioterrorism-Related Conditions.” NEJM 2015; 372: 954-62, https://doi.org/10.1058/NEJMra1409755
Workman S, et al. “Rapid detection of HIV-1 p24 antigen using magnetic immuno-chromatography (MICT),” J. Virol. Methods. 2009; 160:14-21. doi: https://doi.org/10.1016/j.jviromet.2009.04.003
Butler S A, et al. “Detection of early pregnancy forms of human chorionic gonadotropin by home pregnancy test devices.” Clin. Chem. 2001 December:47(12):2131-2136. PMID: 11719477.
Koczula K M, Gallotta A, “Lateral flow assays,” Essays Biochem (2016) 60 (1): 111-120. doi: https//doi.org/10.1042/EBC20150012
Li Z, et al. “Rapid and sensitive detection of protein biomarker using a portable fluorescence biosensor based on quantum dots and a lateral flow test strip.” Anal. Chem. 2010; 82:7008-7014. doi: 10.1021/ac10140Sa.
Song L W, et al. “Rapid fluorescent lateral-flow immunoassay for hepatitis B virus genotyping,” Anal. Chem. 2015; 87:5173-5180. doi: 10.1021/ac504832c.
Venkatraman V., Steckl A. J. “Integrated OLED as excitation light source in fluorescent lateral flow immunoassays,” Biosens. Bioelectron. 2015; 74:150-155. doi: 10.1016/j.bios.2015.06.049.
Liu C, et al. “Lateral flow immunochromatographic assay for sensitive pesticide detection by using Fe3O4 nanoparticle aggregates as color reagents,” Anal. Chem. 2011; 83:6778-8784.
Wang D B, et al. “Detection of Bacillus anthracis spores by super-paramagnetic lateral-flow immunoassays based on ‘Road Closure,’” Biosens. Bioelectron. 2015; 67:608-814. doi: 10.1016/j.bios.2014.09.067.
Mirasoli M, et al. “Development of a chemiluminescence-based quantitative lateral flow immunoassay for on-field detection of 2,4,6-trinitrotoluene,” Anal. Chim. Acta. 2012; 721:167-172. doi: 10.1016/j.aca.2012.01.036.
Maiolini E, et al. “Bisphenol A determination in baby bottles by chemiluminescence enzyme-linked immunosorbent assay, lateral flow immunoassay and liquid chromatography tandem mass spectrometr”. Analyst. 2014:139:318-324. doi: 10.1039/C3AN00552F.
Qiu W, et al. “Carbon nanotube-based lateral flow biosensor for sensitive and rapid detection of DNA sequence,” Biosens. Bioelectron. 2015; 64:367-372. doi: 10.1016/j.bios.2014.09.028.
Koren G, et al. “Drugs that Can Kill a Toddler with One Tablet or Teaspoonful: A 2018 Updated List”. Clin Drug Investig. 2019 February; 39(2):217-220. https//doi.org/10.1007/s40261-018-0726-1
Middlesex-London EMS website (accessed 5/31/2022): https://www.mlems.ca/sites/default/files/7%20Drugs %20that %20can %20kill %20kids %20in % 20a %20single %20pill.pdf
Miller T R, et al. “Incidence and Lethality of Suicidal Overdoses by Drug Class”. JAMA Netw Open. 2020; 3(3):e200607. https://doi.org/10.1001/jamanetworkopen.2020.0607
CDC Morbidity and Mortality Weekly Report (MMWR). “State-Level Economic Costs of Opioid Use Disorder and Fatal Opioid Overdose—United States, 2017.” MMWR 2021 Apr. 16; 70(15):541-6.

### EXAMPLES

### Example 1: Methods of Assay for Detection of Antigen

While MC-ELISA and MLFD's offer certain advantages over traditional ELISA technologies, and while MC-ELISA and MLFD's are well-suited for Fab-DT, more traditional or standard methods of antigen surveillance and detection can still be incorporated into the RB-DT Platform. These include traditional ELISA (Enzyme-Linked Immunosorbent Assay) methods such as competitive ELISA, Sandwich ELISA, and all other variations of ELISA technology. These could be employed in various laboratory-ready and/or field-ready technologies for rapid antigen detection, driving rapid and effective treatment intervention as part of a Fab-DT paradigm.

Additionally, other antibody-based technologies could be employed besides ELISA, including the following, alone or in combination, and with or without modifications:Westem Blot; Immunohistochemistry; Immunocytochemistry; Flow Cytometry and Fluorescence-Activated Cell Sorting (FACS); Immunoprecipitation; or Enzyme-Linked Immunospot (ELISPOT)

Other current or newly developed antibody technologies for antigen detection could be employed in various ways to detect antigens of interest as part of a platform of Fab-DT (whereby diagnosis identifies at once the antigen and the appropriate antibody-based treatment response for that antigen). Any new method of antigen detection using any sort of immune recognition does not antiquate Fab-DT, but rather will serve to enhance it.

Likewise, NA-DT would currently use PCR technology and its various employments. As new methods of DNA or RNA detection or amplification are discovered, and new methods of nucleic acid therapeutic delivery (via plasmid, endosome, liposome, or other nucleic acid delivery system currently known or to be discovered/developed in the future) only enhance the utility of NA-DT and strengthen the concept and clinical scope of RB-DT.

The same is true of covalently interacting anti-toxins, whether these be derived from naturally occurring repositories, from an antidote library (similar to antibody libraries currently in use), from digitized in silico molecular screening or fabrication, or from other existing, emerging, or future technologies. Regardless of the method of identifying or producing covalently bonding anti-toxins, the RB-DT concept remains unchanged, employing such anti-toxins in a diagnostic-therapeutic platform to rapidly identify an offending toxin(s)/agent(s) from a panel of potential candidates in vitro and then using the same diagnostic tool as an antidote in vivo.

1. Antibodies and their Various Derivatives for Detection of Antigen and for Treatment In Vivo
In addition to the disclosure above, antibodies, or other antibody fragments besides Fab fragments could be employed in the Fab-DT paradigm and include (please See FIG. 2): F(ab)₂ fragments, Fab′ fragments, scFv fragments, di-scFv fragments, chemically linked F(ab′)₂ fragments, and BITE (bi-specific T-cell engager) fragments, as well as any derivatives thereof, and any emerging or new antibodies, fragments, or antibody-like products, whether natural or manufactured (e.g., plasmid-driven production in vitro), and regardless of species of origin, post facto humanization of the antibody product or not. Further, the disclosure herein is able to detect post-translational modifications of antibodies or their fragments, whether chemical, enzymatic, or otherwise, do not replace, usurp, or materially change Fab-DT or RB-DT in general.

Additionally, a mixture of these products may employed at the same time, such as already described for Fab-DT, whereby whole antibody is used to PEGylate the antibody to an ELISA plate for purposes of toxin/antigen detection, followed by in vivo administration of the corollary Fab fragment as treatment.

### 2. Considerations for AT-DT and NA-DT

The same concepts outlined above for Fab-DT hold for AT-DT and NA-DT. That is, any variation in source, method, assay, or other measurement, science or art related to the production, employment, administration, or other use of covalently interacting antidotes or nucleic acid tools does not usurp, replace, or materially change AT-DT or NA-DT in concept, with the notion of utilizing a chemical by any available method to rapidly identify an offending toxin or agent, and then in turn posing that same chemical as an antidote in vivo via RB-DT paradigm.

### 3. Other Considerations Regarding Specific Form of RB-DT

Everything said in this section related to Fab-DT, AT-DT, NA-DT, and RB-DT in general, can apply to any existing, emerging or future technology capable of utility in an RB-DT paradigm. The possibility of synergy of movement from diagnosis to antidote in one stroke shall be the distinguishing factor of whether any technology qualifies as RB-DT.

### Example 2: Clinical Scenario: Fab-DT and Antibiotics in Sepsis

Sarah is a 10 year-old female presents to the Emergency Department with multiple episodes of bloody diarrhea the past two days after eating a hamburger from a drive-through restaurant. She is the fourth child over the weekend to present similarly, and all have eaten a hamburger in the past 4 days at a local restaurant. She has a fever of 38.5° C. and commensurate tachycardia. The physician on duty knows that current treatment of a serious enterohemorrhagic food poisoning with the standard antibiotic Rocephin might blow the bacteria apart with release of toxins, worsening the clinical condition. In the event of Shiga-toxin producing E. coli, such release of toxins can precipitate Hemolytic-Uremic Syndrome and renal failure. Using the Fab-DT platform, we can identify the infection as, e.g., Shiga-toxin producing E. coli (STEC) in minutes by ELISA in the hospital's lab. The test demonstrated the presence of STX-1 and STX-2 Shiga toxins in the blood sample sent to the lab. Current culture techniques would have required 2-3 days for STEC identification. The Fab-DT platform simultaneously gives the recipe for treatment: we give the patient a specific antibody Fab fragment identical to the one that bound those toxins from that patient's blood or stool sample in the lab. We now have administered an antibody Fab fragment “sponge,” waiting for the Shiga toxins, already pulling them off human host tissue receptors, and absorbing any release of these toxins caused by the Rocephin we infuse 15 minutes later. Ultimately, the patient avoids severe hemolytic-uremic syndrome and acute renal failure characteristic of STEC, and goes home from the hospital in 3 days instead of 10 days.

What is claimed is:

1. A platform for use in treating the presence of toxins or infectious agents in a sample, wherein:
the sample is a human patient or an environmental sample suspected of having a toxin or infectious agent;
using a treatment modality in an assay for detecting the presence of toxins or infectious agents, wherein an amount of the toxin or infectious agent is capable of being quantified; and
administering the detected treatment modality when the toxin or infectious agent is detected.

2. The platform of claim 1, wherein the treatment modality is an antitoxin.

3. The platform of claim 1, wherein the assay is a nucleic acid-based assay.

4. The platform of claim 1, wherein the assay is an antitoxin-based assay.

5. The platform of claim 2, wherein the antitoxins is enzymatic or nonenzymatic.

6. The platform of claim 1, wherein the platform is an assay.

7. The platform of claim 1, wherein the sample is mechanically or biologically processed or nonprocessed.

8. An assay system for detecting the presence of toxins in a sample, comprised of an assay plate, a population of toxin specific primary antibodies, and a secondary antibody, wherein:
the population of toxin specific primary antibodies are monoclonal;
a single, toxin specific primary antibody from the population of antibodies is affixed to a single well of the assay plate and
the secondary antibody is specific to heavy chain constant regions of each of the toxin specific primary antibodies and the binding is capable of being quantified,
the population of toxin specific primary antibodies are monoclonal;
a single, toxin specific primary antibody from the population of antibodies is affixed to a single well of the assay plate and
the secondary antibody is specific to heavy chain constant regions of each of the toxin specific primary antibodies and the binding is capable of being quantified.

9. The assay system of claim 8 further comprising assay buffer.

10. The assay system of claim 8, wherein the toxin specific primary antibodies are whole antibodies, antigen binding fragments (Fab) or prime antigen binding fragments F(ab′)₂.

11. The assay system of claim 8, wherein the primary antibodies are humanized antibodies or humanized Fab or Fab′₂ fragments.

12. The assay system of either one of claim 9 or claim 10, wherein the primary antibodies are capable of detecting proteins with post-translational modifications.

13. The assay system of claim 8, wherein the toxin specific primary antibodies are affixed to the wells of the assay plate using PEGylation.

14. The assay system of claim 8, wherein the assay plate contains 1-3 wells of the same toxin specific primary antibody.

15. The assay system of claim 8, wherein the bioassay is a modified competitive bioassay.

16. The assay system of claim 8, wherein the secondary antibody competes with toxins in the biological sample for binding to the heavy chain constant regions of the toxin specific primary antibodies.

17. The assay system of claim 8, wherein the secondary antibody binding is quantified using substrates, fluorescence, radiotracers, reaction kinetics, colorimetric changes, or enzyme activity.

18. The assay system of claim 17, wherein secondary antibody binding quantification is inversely proportional to the presence of a toxin in the biological sample.

19. The assay system of claim 8, wherein the toxin specific primary antibodies affixed to the wells recognize distinct epitopes of the same toxin.

20. The assay system of claim 8 wherein the toxin is associated with an infectious agent, virus, or bacteria.

21. The assay system of claim 8, wherein the toxin is associated with a bioterrorism agent, environmental agent, environmental toxin, or non-biological agent.

22. The assay system of claim 8, wherein the toxins are plant or animal based.

23. The platform of claim 1 or the assay system of claim 8, wherein the sample comprises peripheral blood, sera, plasma, ascites, urine, cerebrospinal fluid (CSF), sputum, saliva, bone marrow, synovial fluid, aqueous humor, amniotic fluid, cerumen, broncheoalveolar lavage fluid, semen, prostatic fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, purulent exudate, lavage fluids from sinus cavities, bronchopulmonary aspirates, umbilical cord blood, tissue culture medium, tissue extracts such as homogenized tissue, tumor tissue, cellular extracts, or combinations thereof.

24. The platform of claim 1 or the assay system of claim 8, wherein the sample comprises soil, plants, plant material, water, gas, air, vapors, or condensed vapors.

25. A kit comprising the assay system of claim 8, assay buffer, positive and negative controls, a plate sealant, and instructions.

26. An article for detecting the presence of toxins in a sample, comprising a population of toxin specific primary antibodies, affixed to an assay plate, wherein:
the population of toxin specific primary antibodies are monoclonal; and
a single, toxin specific primary antibody from the population of antibodies is affixed to a single well of the assay plate via PEGylation.

27. A method for detecting the presence of a toxin in a sample comprising
preparing a sample;
passing the sample over an assay plate comprised of toxin specific primary antibodies, affixed to the assay plate;
agitating the assay plate;
removing the sample;
adding a secondary antibody with a quantifiable label and that specifically binds to the heavy chain constant region of the primary antibody;
agitating the assay plate;
removing the secondary antibody; and
quantifying the binding of the secondary antibody to the heavy chain constant region wherein secondary antibody binding to the heavy chain constant region is inversely proportional to the quantity of toxin in the sample.

28. A method of treating a subject suffering from toxin exposure, wherein the method of claim 27 is used to identify the toxin and one or more monoclonal antibodies to be used as therapeutic modalities.

29. The method of claim 28, wherein the monoclonal antibody is administered intravenously, intramuscularly, subcutaneously, or transdermally.

30. The assay system of claim 8, wherein the assay is an Enzyme-Linked Immunosorbent Assay (ELISA) or sandwich ELISA.

31. The assay system of claim 8, wherein the assay is an antibody-based technology consisting of a western blot, immunohistochemistry, immunocytochemistry, Flow Cytometry and Fluorescence-Activated Cell Sorting (FACS), or an Immunoprecipitation Enzyme-Linked Immunospot (ELISPOT)

32. A lateral flow device for detecting the presence of toxins in a sample, wherein the lateral flow device is comprised of an absorbent pad, nitrocellulose membrane, and antibody coated latex microbeads.

33. The lateral flow device of claim 32, wherein the absorbent pad is engineered to filter bacteria and other contaminants.

34. The lateral flow device of claim 32 wherein the nitrocellulose membrane has a pore size of 0.5 to 30 micrometers.

35. The nitrocellulose membrane of claim 34, wherein the nitrocellulose membrane has a polyester backing.

36. The lateral flow device of claim 32, wherein the latex microbeads are coated with a toxin specific primary antibody.

37. The lateral flow device of claim 32, wherein the latex microbeads are coated with a population of toxin specific primary antibodies.

38. The lateral flow device of claim 37, wherein the toxin specific primary antibodies are monoclonal.

39. The lateral flow device of claim 32, wherein the latex microbeads produce a colorimetric response upon binding of the target antigen.

40. The lateral flow device of claim 32, wherein the latex microbeads produced a radioactive signal upon binding of the target antigen.

41. The lateral flow device of claim 32, wherein the latex microbeads produced a photometric signal upon binding of the target antigen.

42. The lateral flow device of claim 32, wherein antibodies for a second toxin epitope bind the colored latex microbead-toxin complex thereby generating a colorimetric signal.

43. The lateral flow device of claim 32, comprising an air sampling apparatus.

44. A method for treating the presence of an infectious agent or toxin in a sample comprising
preparing a sample;
contacting the sample with the lateral flow device absorbent pad of device of claim 25
determining the presence or absence of a toxin by observing a colorimetric indicator change in the lateral flow device; and
wherein one or more toxins are present,
administering the appropriate treatment modality against the toxin or infectious agent.

45. The method of claim 44, wherein the sample comprises peripheral blood, sera, plasma, ascites, urine, cerebrospinal fluid (CSF), sputum, saliva, bone marrow, synovial fluid, aqueous humor, amniotic fluid, cerumen, broncheoalveolar lavage fluid, semen, prostatic fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, purulent exudate, lavage fluids from sinus cavities, bronchopulmonary aspirates, umbilical cord blood, tissue culture medium, tissue extracts such as homogenized tissue, tumor tissue, cellular extracts, or combinations thereof.

46. The method of claim 44, wherein the sample comprises soil, plants, plant material, water, gas, air, vapors, or condensed vapors.

47. A kit comprising the lateral flow device of claim 32 for detecting and identifying the presence of the toxin or infectious agent.

48. An assay system for detecting the presence of toxins in a sample, comprised of an assay plate, a population of toxin specific nucleic acid primers, and associated probes, wherein:
the population of toxin specific nucleic acid primers are directed against sequences of an infecting agent or toxin;
polymerase chain reaction is use to amplify the sequence in a sample of interest and
the sequence of an infecting agent or toxin is capable of being quantified.

49. An assay system for detecting the presence of toxins in a sample, comprised of an assay plate and a population of antitoxins, wherein:
the population of antitoxins covalently bind a toxin in a sample;
and the binding between antitoxins and toxins is capable of being quantified.
